(19) United States
(12) Patent Application Publication
(10) Pub. No.: US 2009/0280134 A1
(43) Pub. Date: Nov. 12, 2009

(54) COMPOSITIONS AND METHODS FOR INHIBITING TOXIN A FROM CLOSTRIDIUM DIFFICILE

(75) Inventors: Jan Holgersson, Huddinge (SE); Anki Gustafsson, Tullinge (SE)

Correspondence Address: MINTZ, LEVIN, COHN, FERRIS, GLOVSKY AND POPEO, P.C, ONE FINANCIAL CENTER, BOSTON, MA, 02111, US

(73) Assignee: RECOPHARMA AB, Huddinge (SE)

(21) Appl. No.: 12/463,951

(22) Filed: May 11, 2009

Related U.S. Application Data
(60) Provisional application No. 61/051,883, filed on May 9, 2008.

(51) Int. Cl. A61K 39/08

(52) U.S. Cl. 424/183.1; 424/185.1

(57) ABSTRACT

The present invention provides compositions and methods for treating or preventing infection by Toxin A producing bacteria.

### REFERENCE TO RELATED APPLICATIONS

This non-provisional application claims priority under 35 U.S.C. § 119(e) to U.S. Provisional Application No. 61/051,883 filed May 9, 2008, the contents of which are hereby incorporated by reference in its entirety.

### FIELD OF THE INVENTION

The invention relates to generally to compositions and methods for treating or preventing infection by Toxin A produced by Clostridium difficile, and more particularly to compositions including fusion polypeptides comprising carbohydrate epitopes that inhibit Toxin A.

### BACKGROUND OF THE INVENTION

Toxin A of Clostridium difficile is a 308 kDa protein with seven putative binding sites for Galα1,3Galβ1,4GlcNAc, presumably both lipid- and protein-bound. The binding pocket may tolerate some modifications, such as fucosylation, as binding also to Le^{x} and Le^{y} structures is accepted. Upon binding to the host cell surface, toxin A is endocytosed. It glucosylates Rho proteins in the cytosol, thereby disrupting their normal functions including regulation of the epithelial cell barrier. C. difficile is an opportunistic pathogen and the most common cause of antibiotic-associated diarrhoea. Antibiotics disturb the normal bacterial flora of the intestine, allowing for C. difficile overgrowth.

### SUMMARY OF THE INVENTION

The invention is based in part on the discovery that carbohydrate epitopes that mediate (i.e., block, inhibit) the binding of Toxin A to a host cell surface can be specifically expressed at high density and by different core saccharide chains on mucin-type protein backbones. The polypeptides are referred to herein as αGal fusion proteins or αGal polypeptides. These recombinant, heavily glycosylated proteins carrying ample O-linked glycans capped with carbohydrate determinants with known bacterial toxin-binding activity can act as decoys, and as such specifically prevent (e.g., sterically inhibit) bacterial toxin infection in for example, the respiratory or the gastrointestinal tracts. The fusion proteins have low toxicity and low risk of inducing bacterial resistance to the drugs.

In one aspect, the invention provides a fusion polypeptide that includes a first polypeptide that carries the Galα1,3Gal carbohydrate epitope, operably linked to a second polypeptide. The first polypeptide is multivalent for these epitopes. The first polypeptide is, for example, a mucin polypeptide such as PSGL-1 or portion thereof. Preferably, the mucin polypeptide is the extracellular portion of PSGL-1.

In one particular embodiment, the fusion polypeptide of the invention comprises a glycan repertoire including one or more sequences selected from Hex-HexNol-HexN-Hex-Hex, NeuAc-Hex-HexNol-HexN-Hex-Hex and NeuGc-Hex-HexNol-HexN-Hex-Hex, or any fragment, segment, or portion of said sequences. Alternatively, the fusion polypeptide of the invention comprises a glycan repertoire including one or more of the sequences shown in Table 2, or any fragment, segment or portion of said sequences.

The second polypeptide comprises at least a region of an immunoglobulin polypeptide. For example, the second polypeptide comprises a region of a heavy chain immunoglobulin polypeptide. Alternatively, the second polypeptide comprises the Fc region of an immunoglobulin heavy chain.

The fusion polypeptide is a multimer. Preferably, the fusion polypeptide is a dimer.

Also included in the invention is a nucleic acid encoding the αGal fusion polypeptide, as well as a vector containing αGal fusion polypeptide-encoding nucleic acids described herein, and a cell containing the vectors or nucleic acids described herein. Optionally, the vector further comprises a nucleic acid encoding one or more glycotransferases necessary for the synthesis of the desired carbohydrate epitope. For example, the vector contains a nucleic acid encoding a α1,3 galactosyltransferase and a nucleic acid encoding a β1,6,-N-acetylglucosaminyltransferase.

In another aspect, the invention provides a method of inhibiting (e.g., decreasing) the binding of Toxin A to a cell surface. Binding is inhibited by contacting Toxin A and/or Toxin A producing bacteria with the αGal fusion polypeptide of the invention. The invention also features methods of preventing or alleviating a symptom of Toxin A producing bacterial infection or a disorder associated with Toxin A producing bacterial infection in a subject by identifying a subject suffering from or at risk of developing Toxin A producing bacterial infection and administering to the subject the fusion polypeptide of the invention. The bacteria is for example, Clostridium difficile (C. difficile).

The subject is a mammal such as human, a primate, mouse, rat, dog, cat, cow, horse, pig. The subject is suffering from or at risk of developing a Toxin A producing bacterial infection or a disorder associated with a Toxin A producing bacterial infection. A subject suffering from or at risk of developing a Toxin A producing bacterial infection or a disorder associated with a Toxin A producing bacterial infection is identified by methods known in the art

Also included in the invention are pharmaceutical compositions that include the fusion polypeptides of the invention.

Also provided by the invention are methods of producing the αGal fusion polypeptide of the invention. Fusion polypeptides are produced by providing a cell containing a nucleic acid encoding a mucin polypeptide operably linked to a nucleic acid encoding at least a portion of an immunoglobulin polypeptide; a nucleic acid encoding an α1,3 galactosyltransferase polypeptide; and a nucleic acid encoding a β1,6,-N-acetylglucosaminyltransferase polypeptide. Alternatively, fusion polypeptides are produced by introducing to a cell (e.g., transfection or transformation) a nucleic acid encoding a mucin polypeptide operably linked to a nucleic acid encoding at least a portion of an immunoglobulin polypeptide; a nucleic acid encoding an α1,3 galactosyltransferase polypeptide; and a nucleic acid encoding a β1,6,-N-acetylglucosaminyltransferase polypeptide. The cell is cultured under conditions that permit production of the fusion polypeptide and the fusion polypeptide is isolated from the culture. Fusion polypeptides are isolated by methods known in the art. For example, the fusion polypeptides are isolated using Protein A or Protein G chromatography.

The cell is a eukaryotic cell, or a prokaryotic cell, e.g. a bacterial cell. A eukaryotic cell is, for example, a mammalian cell, an insect cell or a yeast cell. Exemplary eukaryotic cells include a CHO cell, a COS cell or a 293 cell.

The mucin polypeptide is for example PSGL-1. Preferably, the mucin polypeptide is the extracellular portion of PSGL-1. In preferred embodiments, the second polypeptide comprises at least a functional region of an immunoglobulin polypeptide. For example, the second polypeptide comprises a region of a heavy chain immunoglobulin polypeptide. Alternatively, the second polypeptide comprises the FC region of an immunoglobulin heavy chain.

The fusion polypeptide is a multimer. Preferably, the fusion polypeptide is a dimer.

Also included in the invention is a nucleic acid encoding an αGal fusion polypeptide, as well as a vector containing an αGal fusion polypeptide-encoding nucleic acids described herein, and a cell containing the vectors or nucleic acids described herein. Alternatively the vector further comprises a nucleic acid encoding a an α1,3 galactosyltransferase and/or a core 2 β1,6-N-acetylglusosaminyltransferase. The invention also includes host cell, e.g. CHO cells genetically engineered to express the αGal fusion polypeptide.

Also included in the invention are pharmaceutical compositions that include the αGal fusion polypeptides.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 shows photographs of SDS-PAGE of proteins isolated from supernatants of COS cells transfected with vector alone (CDM8), PSGL1/mIgG_{2b}, or PSGL1/mIgG_{2b} and porcine α1,3GT expression plasmids. These were subsequently probed with peroxidase-conjugated Bandeireia simplicifolia isolectin B₄ lectin and visualized by chemiluminescens to detect Galα1,3 Gal epitopes on immunopurified proteins.

FIG. 2A is a bar chart showing quantification by anti-mouse IgG Fc ELISA of the PSGL1/mIgG_{2b} fusion protein concentration in increasing volumes of transfected COS cell supernatants before and after absorption on 50 μl of anti-mouse IgG agarose beads. Triplicate samples were analyzed.

FIG. 2B is a photograph of a gel showing the PSGL1/mIgG_{2b} fusion protein concentration in increasing volumes of transfected COS cell supernatants

FIG. 3 is a photograph of a SDS-PAGE gelof immunoaffinity purified human IgG, IgM, and IgA. Four micrograms of each sample were run under reducing and non-reducing conditions, and proteins were visualized by silver staining.

FIG. 4 is a photograph of a Western blot depicting PSGL-1/mIgG_{2b} fusion proteins immunoaffinity purified from supernatants of CHO-K1, COS and 293T cells stably transfected with the PSGL-1/mIgG_{2b} cDNA alone (−) or together with the porcine of α1,3 galactosyltransferase cDNA (+).

FIG. 5 is a bar chart showing the relative α-Gal epitope density on PSGL-1/mIgG_{2b} expressed by CHO-K1, COS, and 293T cells.The relative α-Gal epitope density on P-selectin glycoprotein ligand-1-mouse immunoglobulin Fc fusion proteins (PSGL-1/mIgG_{2b}) produced in CHO-K1, COS or 293T without (white bars) or with (black bars) co-expression of the pig α1,3galactosyltransferase (GalT) and, for CHO-K1, the core 2 β1,6 N-acetylglucosaminyl transferase (C2 GnTI) (grey bar).

FIG. 6 is a photograph of a Western blot analysis of PSGL-1/mIgG_{2b} fusion protein immunoaffinity purified from supernatants of stably transfected CHO-K1 cells.

FIG. 7 shows photographs of SDS-PAGE and Western blot analysis of PSGL-1/mIgG_{2b} purified by affinity chromatography and gel filtration.

FIG. 8 is an illustration depicting electrospray ion trap mass spectrometry analysi of O-glycans released from PSGL-1/mIgG_{2b} made in CHO clone 5L4-1.

FIG. 9 is an illustration depicting electrospray ion trap mass spectrometry of O-glycans released from PSGL-1/mIgG_{2b} made in CHO clone C2-1-9.

FIG. 10 is a series of illustrations depicting MS/MS analyses of the predominant peak seen in the mother spectra of O-glycans released from PSGL-1/mIgG_{2b} made in CHO clone C2-1-9. DETAILED DESCRIPTION

The invention is based in part in the discovery that the carbohydrate epitope Galα1, 3Gal (αGal) can be specifically expressed at high density and by different core saccharides chains on mucin-type protein backbones. More particularly, the invention is based upon the surprising discovery that expression of αGal epitopes of mucin-type protein backbones is dependent upon the cell line expressing the polypeptide. Moreover, the glycan repertoire of the mucin can be modified by co-expresion of exogenous α1,3 galactosyltransferase and a core 2 branching enzyme. This modification results in a higher density of αGal eptiopes and an increased binding or removal (i.e., absorption) of anti-αGal antibodies as compared to free saccharides, αGal determinants linked to solid phase, or cells transfected with α1,3 galactosyltransferase alone.

Transient transfection of a PSGL-1/mIgG_{2b} fusion protein and porcine α1,3galactosyltransferase (α1,3GalT) in COS cells results in a dimeric fusion protein heavily substituted with α-Gal epitopes. The fusion protein has approximately 20 times higher (on a carbohydrate molar basis) terminal α-Gal epitopes per dimer than pig thyroglobulin immobilized on agarose beads, and 5,000 and 30,000 times higher than Galα1,3Gal-conjugated agarose and macroporous glass beads, respectively.

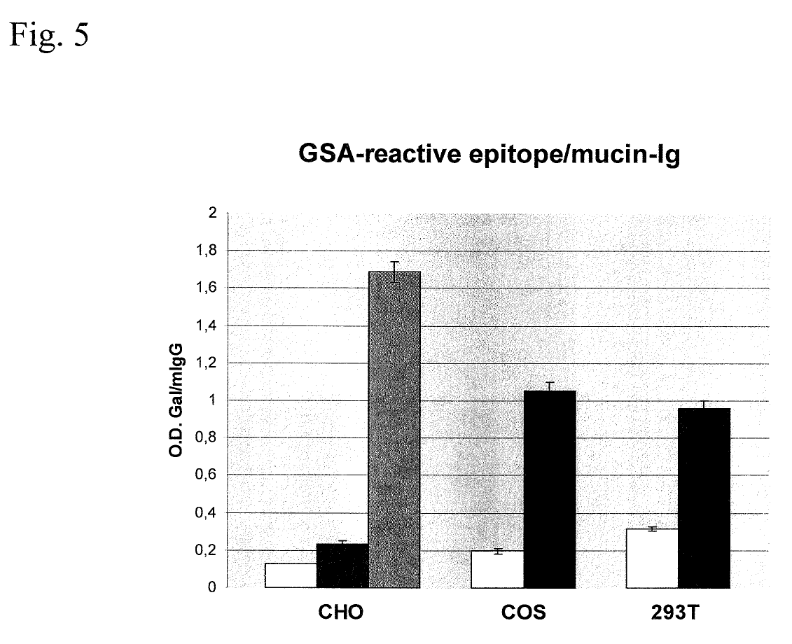

To investigate the importance of the host cell for α-Gal epitope density on PSGL-1/mIgG_{2b}, the protein, together with the porcine α1,3GalT, was stably expressed in CHO, COS and 293T cells. The level of α-Gal substitution on PSGL-1/mIgG_{2b} was dependent on the host cell. PSGL-1/mIgG_{2b} made in COS cells exhibited a 5.3-fold increase in the relative O.D. (GSA-reactivity/anti-mouse IgG reactivity) compared to PSGL-1/mIgG_{2b} made in COS without the α1,3GalT (FIG. 5). Similarly, PSGL-1/mIgG_{2b} made in 293T cells exhibited a 3.1-fold increase in the relative O.D. In contrast, PSGL-1/mIgG_{2b} made in CHO cells exhibited only a 1.8-fold increase (FIG. 5).

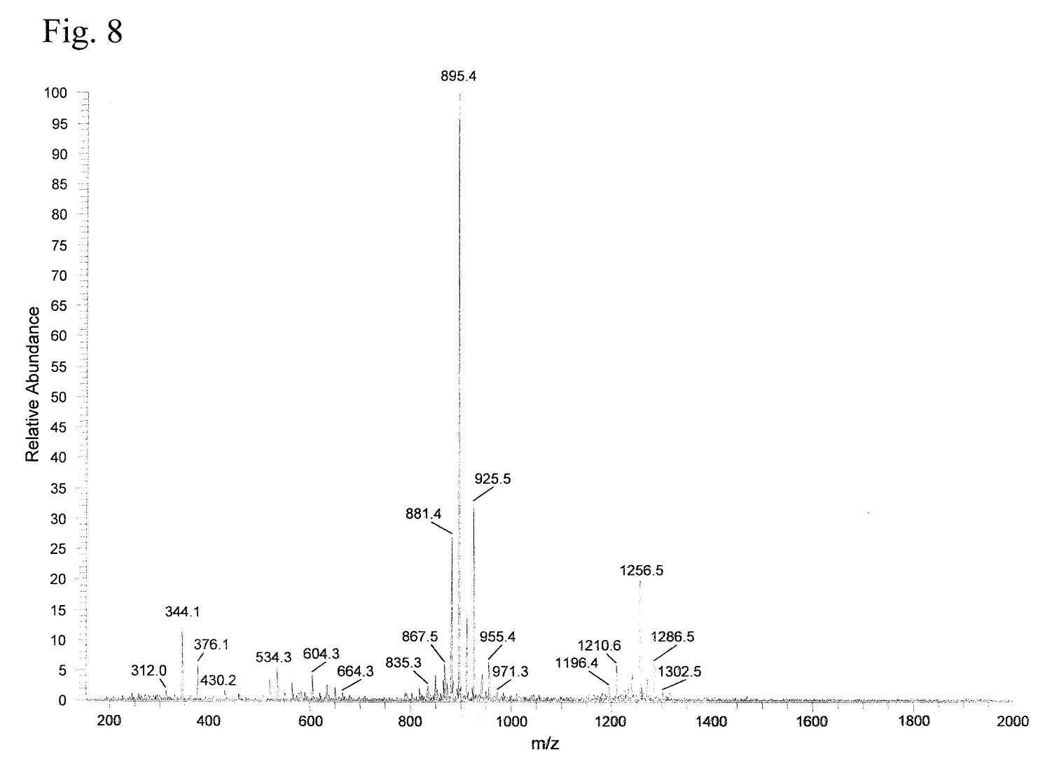

Surprisingly, co-expression of a core 2 β1,6 GlcNAc transferase (C2 GnTI) in CHO cells improved PSGL-1/mIgG_{2b} α-Gal epitope density. Moreover, PSGL-1/mIgG_{2b} expressed in CHO cells together with the porcine α1,3GalT and the C2 GnTI carried three different O-glycans with sequences consistent with terminal Gal-Gal. (Table 2). In contrast, no terminal Gal-Gal epitopes were detected on O-glycans on PSGL-1/mIgG_{2b} expressed in CHO cels without the C2 GnTI. As shown in FIG. 5, the level of α-Gal epitopes on the fusion protein produced in CHO cells expressing both exogenous C2 GnTI and α1,3GalT was strikingly increased, exceeding the α-Gal epitope levels on the fusion protein made in COS and 293T cells expressing only exogenous α1,3GalT. Mass spectrometry confirmed that, the increased α-Gal epitope density was due to core 2 branching and lactosamine extensions on O-glycans of PSGL-1/mIgG_{2b} made in CHO cells engineered to express both C2 GnTI and α1,3GalT (FIG. 8, 9 and Table II). The structural analysis of the O-glycans expressed on CHO cells co-expressing the α1,3GalT and the C2 GnTI also showed that the α-Gal epitope was expressed on three different oligosaccharides (FIG. 8 and Table II).

### Inhibition of Toxin A

The invention is also based, in part, in the discovery that carbohydrate epitopes that mediate (i.e., block, inhibit) the binding activity of Toxin A can be specifically expressed at high density on glycoproteins, e.g., mucin-type protein backbones. This higher density of carbohydrate epitopes results in an increased valancy and affinity compared to monovalent oligosaccharides and wild-type, e.g. native non recombinantly expressed glycoproteins.

Toxin A producing bacteria (e.g., C. difficile) bind to host cells via the specific cell surface glycoplipids Galα1,3Galβ1,4GlcNAc. Upon binding to the surface of a host cell, the toxin is internalized and glucosylates Rho proteins in the cytosol, thereby disrupting their normal functions including regulation of the epithelial cell barrier resulting in diarrhea.

The αGal fusion proteins of the invention are useful in mediating (i.e., blocking, inhibiting) the binding interaction between Toxin A and a host cell surface. The epitopes are terminal, i.e, at the terminus of the glycan. The αGal fusion protein inhibits 10%, 20%, 30%, 40%, 50%, 60%, 70%, 80%, 90%, 95%, 98% or 100% of the binding of Toxin A to a cell surface. The αGal fusion peptide is more efficient on a carbohydrate molar basis in the binding activity of inhibiting Toxin A as compared to free saccharrides. The αGal fusion peptide inhibits 2, 4, 10, 20, 50, 80, 100 or more-fold greater amount of toxin as compared to an equivalent amount of free saccharrides.

### Fusion Polypeptides

In various aspects the invention provides fusion proteins that include a first polypeptide containing at least a portion of a glycoprotein, e.g. a mucin polypeptide linked to a second polypeptide. As used herein, a “fusion protein” or “chimeric protein” includes at least a portion of a mucin polypeptide operatively linked to a non-mucin polypeptide. A “non-mucin polypeptide” refers to a polypeptide of which at least less than 40% of its mass is due to glycans.

A “mucin polypeptide” refers to a polypeptide having a mucin domain. The mucin polypeptide has one, two, three, five, ten, twenty or more mucin domains. The mucin polypeptide is any glycoprotein characterized by an amino acid sequence subsitited with O-glycans. For example a mucin polypeptide has every second or third amino acid being a serine or threonine. The mucin polypeptide is a secreted protein. Alternatively, the mucin polypeptide is a cell surface protein.

Mucin domains are rich in the amino acids threonine, serine and proline, where the oligosaccharides are linked via N-acetylgalactosamine to the hydroxy amino acids (O-glycans). A mucin domain comprises or alternatively consists of an O-linked glycosylation site. A mucin domain has 1, 2, 3, 5, 10, 20, 50, 100 or more O-linked glycosylation sites. Alternatively, the mucin domain comprises or alternatively consists of a N-linked glycosylation site. A mucin polypeptide has 50%, 60%, 80%, 90%, 95% or 100% of its mass due to the glycan. A mucin polypeptide is any polypeptide encode for by a MUC genes (i.e., MUC1, MUC2, MUC3, MUC4, MUC5a, MUC5b, MUC5c, MUC6, MUC11, MUC12, etc.). Alternatively, a mucin polypeptide is P-selectin glycoprotein ligand 1 ( PSGL-1), CD34, CD43, CD45, CD96, GlyCAM-1, MAdCAM-1, red blood cell glycophorins, glycocalicin, glycophorin, sialophorin, leukosialin, LDL-R, ZP3, and epiglycanin. Preferably, the mucin is PSGL-1. PSGL-1 is a homodimeric glycoprotein with two disulfide-bonded 120 kDa subunits of type 1 transmembrane topology, each containing 402 amino acids. In the extracellular domain there are 15 repeats of a 10-amino acid consensus sequence that contains 3 or 4 potential sites for addition of O-linked oligosaccharides. In one embodiment, the 10-amino acid consensus sequence is A(I) Q T T Q(PAR) P(LT) A(TEV) A(PG) T(ML) E (SEQ ID NO: 1). In another embodiment, the 10-amino acid consensus sequence is A Q(M) T T P(Q) P(LT) A A(PG) T(M) E (SEQ ID NO: 34). PSGL-1 is predicted to have more than 53 sites for O-linked glycosylation and 3 sites for N-linked glycosylation in each monomer.

The mucin polypeptide contains all or a portion of the mucin protein. Alternatively, the mucin protein includes the extracellular portion of the polypeptide. For example, the mucin polypeptide includes the extracellular portion of PSGL-1 or a portion thereof (e.g., amino acids 19-319 disclosed in GenBank Accession No. A57468). The mucin polypeptide also includes the signal sequence portion of PSGL-1 (e.g., amino acids 1-18), the transmembrane domain (e.g., amino acids 320-343), and the cytoplamic domain (e.g., amino acids 344-412).

Within an αGal fusion protein of the invention the mucin polypeptide corresponds to all or a portion of a mucin protein. For example, an αGal fusion protein cotains at least a portion of a mucin protein. “At least a portion” is meant that the mucin polypeptide contains at least one mucin domain (e.g., an O-linked glycosylation site). Optionally, the mucin protein comprises the extracellular portion of the polypeptide. For example, the mucin polypeptide comprises the extracellular portion of PSGL-1.

The mucin polypeptide is decorated with a glycan repertoire as shown in Table. 2. For example the mucin polypeptide has one, two, three, four, five or more the carbohydrate sequences recited in Table 2. For example the mucin polypeptide has the glycan repertoire including Hex-HexNol-HexN-Hex-Hex; NeuAc-Hex-HexNol-HexN-Hex-Hex; and NeuGc-Hex-HexNol-HexN-Hex-Hex. The mucin polypeptide has one, two, three, four, five or more terminal αGal sugars. Preferably, the terminal sugars are expressed on two, three, four, five or more different oligosaccarides. Optionally, the mucin includes N-acetyl neuraminic acid, N-glycolyl neuraminic acid, and/or sialic acid. Additionally, the oligosaccarides of the mucin includes core 2 braching, core 1 branching, and lactosamine extensions.

The first polypeptide is glycosylated by one or more transferases. The transferase is exogenous. Alternatively, the transferase is endogenous. The first polypeptide is glycosylated by 2, 3, 5 or more transferases. Glycosylation is sequential or consecutive. Alternatively glycosylation is concurrent or random, i.e., in no particular order. For example the first polypeptide is glycosylated by an α1,3 galactosyltransferase. Suitable sources for α1,3 galactosyltransferase include GenBank Accession Nos. AAA73558, L36150, BAB30163, AK016248, E46583 or P50127 and are incorporated herein by reference in their entirety. Alternatively, the first polypeptide is glycosylated by core 2 branching enzyme or an N acetylglucosaminyltransferase such as a β 1,6 N-acetylglucosaminyltransferase. Suitable sources for a β1,6 N-acetylglucosaminyltransferase include GenBank Accession Nos. CAA796 10, Z 19550, BAB66024, AP001515, AJ420416.1, AK313343.1, AL832647.2, AY196293.1, BC074885.2, BC074886, BC109101, BC109102.1, M97347.1, BAG36146.1, CAD89956.1, AAH74885.1, AAH74886.1, AAI109102.1, AAI09103.1, AAA35919.1, AAH17032, 095395, NP_{—}004742, EAW77572, NP_{—}004742.1, BC017032, AF102542.1, AAD10824.1, AF038650.1, NM_{—}004751.2, Q9P109, NP_{—}057675, EAW95751, AF132035.1, AAF63156.1, and NP_{—}057675.1. Preferably, the firstpolypeptide is glycosylated by both an α1,3 galactosyltransferase and a β1,6 N-acetylglucosaminyltransferase. The first polypeptide contains greater than 40%, 50%, 60%, 70%, 80%, 90% or 95% of its mass due to carbohydrate.

Within the fusion protein, the term “operatively linked” is intended to indicate that the first and second polypeptides are chemically linked (most typically via a covalent bond such as a peptide bond) in a manner that allows for O-linked glycosylation of the first polypeptide. When used to refer to nucleic acids encoding a fusion polypeptide, the term operatively linked means that a nucleic acid encoding the mucin polypeptide and the non-mucin polypeptide are fused in-frame to each other. The non-mucin polypeptide can be fused to the N-terminus or C-terminus of the mucin polypeptide.

Optionally, the αGal fusion protein is linked to one or more additional moieties. For example, the αGal fusion protein is linked to a GST fusion protein in which the αGal fusion protein sequences are fused to the C-terminus of the GST (i.e., glutathione S-ERROR) sequences. Such fusion proteins can facilitate the purification of αGal fusion protein. Alternatively, the αGal fusion protein is additionally linked to a solid support. Various solid supports are known to those skilled in the art. For example, the αGal fusion protein is linked to a particle made of, e.g., metal compounds, silica, latex, polymeric material; a microtiter plate; nitrocellulose, or nylon or a combination thereof. The αGal fusion proteins linked to a solid support are used as as a diagnostic or screening tool for bacterial producing shiga toxin and shiga-like toxin infection.

The fusion protein includes a heterologous signal sequence (i.e., a polypeptide sequence that is not present in a polypeptide encoded by a mucin nucleic acid) at its N-terminus. For example, the native mucin signal sequence can be removed and replaced with a signal sequence from another protein. In certain host cells (e.g., mammalian host cells), expression and/or secretion of polypeptide can be increased through use of a heterologous signal sequence.

A chimeric or fusion protein of the invention can be produced by standard recombinant DNA techniques. For example, DNA fragments coding for the different polypeptide sequences are ligated together in-frame in accordance with conventional techniques, e.g. by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, for example, Ausubel et al. (eds.) CURRENT PROTOCOLS IN MOLECULAR BIOLOGY, John Wiley & Sons, 1992). Moreover, many expression vectors are commercially available that encode a fusion moiety (e.g., an Fc region of an immunoglobulin heavy chain). A PSGL-1 encoding nucleic acid can be cloned into such an expression vector such that the fusion moiety is linked in-frame to the immunoglobulin protein. An exemplary PSGL-1 expression vector include SEQ ID NO:21

An αGal fusion polypeptides exist as oligomers, such as dimers, trimers or pentamers. Preferably, the αGal fusion polypeptide is a dimer.

The first polypeptide, and/or nucleic acids encoding the first polypeptide, is constructed using mucin encoding sequences are known in the art. Suitable sources for mucin polypeptides and nucleic acids encoding mucin polypeptides include GenBank Accession Nos. NP663625 and NM145650, CAD10625 and AJ417815, XP 140694 and XM140694, XP006867 and XM006867 and NP00331777 and NM009151 respectively, and are incorporated herein by reference in their entirety.

Alternatively, the mucin polypeptide moiety is provided as a variant mucin polypeptide having an alteration in the naturally-occurring mucin sequence (wild type) that results in increased carbohydrate content (relative to the non-variant or wild type sequence). As used herein, an alteration in the naturally-occurring (wild type) mucin sequence includes one or more one or more substitutions, additions or deletions into the nucleotide and/or amino acid sequence such that one or more amino acid substitutions, additions or deletions are introduced into the encoded protein. Alterations can be introduced into the naturally-occurring mucin sequence by standard techniques, such as site-directed mutagenesis and PCR-mediated mutagenesis.

For example, the variant mucin polypeptide comprised additional O-linked glycosylation sites compared to the wild-type mucin. Alternatively, the variant mucin polypeptide comprises an amino acid sequence alteration that results in an increased number of serine, threonine or proline residues as compared to a wild type mucin polypeptide. This increased carbohydrate content can be assessed by determining the protein to carbohydrate ratio of the mucin by methods known to those skilled in the art. Alternatively, the mucin polypeptide moiety is provided as a variant mucin polypeptide having alterations in the naturally-occurring mucin sequence (wild type) that results in a mucin sequence with more O-glycosylation sites or a mucin sequence preferably recognized by peptide N-acetylgalactosaminyltransferases resulting in a higher degree of glycosylation.

In some embodiments, the mucin polypeptide moiety is provided as a variant mucin polypeptide having alterations in the naturally-occurring mucin sequence (wild type) that results in a mucin sequence more resistant to proteolysis (relative to the non-mutated sequence).

The first polypeptide includes full-length PSGL-1. Alternatively, the first polypeptide comprise less than full-length PSGL-1 polypeptide, e.g., a functional fragment of a PSGL-1 polypeptide. For example the first polypeptide is less than 400 contiguous amino acids in length of a PSGL-1 polypeptide, e.g., less than or equal to 300, 250, 150, 100, or 50, contiguous amino acids in length of a PSGL-1 polypeptide, and at least 25 contiguous amino acids in length of a PSGL-1 polypeptide. The first polypeptide is, for example, the extracellular portion of PSGL-1, or includes a portion thereof. Exemplary PSGL-1 polypeptide and nucleic acid sequences include GenBank Access No: XP006867; XM006867; XP140694 and XM140694.

The second polypeptide is preferably soluble. The second polypeptide includes a sequence that facilitates association of the αGal fusion polypeptide with a second mucin polypeptide. Preferably, the second polypeptide includes at least a region of an immunoglobulin polypeptide. “At least a region” is meant to include any portion of an immunoglobulin molecule, such as the light chain, heavy chain, FC region, Fab region, Fv region or any fragment thereof. Immunoglobulin fusion polypeptide are known in the art and are described in e.g. U.S. Pat. Nos. 5,516,964; 5,225,538; 5,428,130; 5,514,582; 5,714,147; and 5,455,165.

The second polypeptide comprises a full-length immunoglobulin polypeptide. Alternatively, the second polypeptide comprise less than full-length immunoglobulin polypeptide, e.g. a heavy chain, light chain, Fab, Fab₂, Fv, or Fc. Preferably, the second polypeptide includes the heavy chain of an immunoglobulin polypeptide. More preferably the second polypeptide includes the Fc region of an immunoglobulin polypeptide.

In another aspect of the invention the second polypeptide has less effector function that the effector function of a Fc region of a wild-type immunoglobulin heavy chain. Fc effector function includes for example, Fc receptor binding, complement fixation and T cell depleting activity. (see for example, U.S. Pat. No. 6,136,310) Methods of assaying T cell depleting activity, Fc effector function, and antibody stability are known in the art. In one embodiment the second polypeptide has low or no affinity for the Fc receptor. In an alternative embodiment, the second polypeptide has low or no affinity for complement protein C1q.

Another aspect of the invention pertains to vectors, preferably expression vectors, containing a nucleic acid encoding mucin polypeptides, or derivatives, fragments, analogs or homologs thereof. In various aspects the vector contains a nucleic acid encoding a mucin polypeptide operably linked to an nucleic acid encoding an immunoglobulin polypeptide, or derivatives, fragments analogs or homologs thereof. Additionally, the vector comprises a nucleic acid encoding a α1,3 galactosyltransferase, a core 1,6,-N-actetylglucosaminyltransferase or any combination thereof. The transferase facilitates the addition of αGal determinants on the peptide backbone of the mucin portion of the αGal fusion protein. Exemplary vectors include SEQ ID NO:1, 11 or 21. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to include promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., ABO fusion polypeptides, mutant forms of ABO fusion polypeptides, etc.).

The recombinant expression vectors of the invention can be designed for expression of αGal fusion polypeptides in prokaryotic or eukaryotic cells. For example, αGal fusion polypeptides can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g. Gottesman, GENE EXPRFSSION TECHNOLOGY: METHODS IN ENZYMOLOGY 185, Academic Press, San Diego, Calif (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the αGal fusion polypeptide expression vector is a yeast expression vector. Examples of vectors for expression in yeast Saccharomyces cerivisae include pYepSecl (Baldari, et al., 1987. EMBO J 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, αGal fusion polypeptide can be expressed in insect cells using baculovirus expression vectors. Baculovirus vectors available for expression of proteins in cultured insect cells (e.g., SF9 cells) include the pAc series (Smith, et al., 1983. MoL CelL BioL 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, αGal fusion polypeptides is expressed in bacterial cells such as E. coli, insect cells, yeast or mammalian cells (such as human, Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding glycoprotein Ibα fusion polypeptides or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) αGal fusion polypeptides. Accordingly, the invention further provides methods for producing αGal fusion polypeptides using the host cells of the invention. In one embodiment, the method comprises culturing the host cell of invention (into which a recombinant expression vector encoding αGal fusion polypeptides has been introduced) in a suitable medium such that αGal fusion polypeptides is produced. In another embodiment, the method further comprises isolating αGal polypeptide from the medium or the host cell.

The αGal fusion polypeptides may be isolated and purified in accordance with conventional conditions, such as extraction, precipitation, chromatography, affinity chromatography, electrophoresis or the like. For example, the immunoglobulin fusion proteins may be purified by passing a solution through a column which contains immobilized protein A or protein G which selectively binds the Fc portion of the fusion protein. See, for example, Reis, K. J., et al., J. Immunol. 132:3098-3102 (1984); PCT Application, Publication No. WO87/00329. The fusion polypeptide may then be eluted by treatment with a chaotropic salt or by elution with aqueous acetic acid (1 M).

Alternatively, αGal fusion polypeptides according to the invention can be chemically synthesized using methods known in the art. Chemical synthesis of polypeptides is described in, e.g., Peptide Chemistry, A Practical Textbook, Bodasnsky, Ed. Springer-Verlag, 1988; Merrifield, Science 232: 241-247 (1986); Barany, et al, Intl. J. Peptide Protein Res. 30: 705-739 (1987); Kent, Ann. Rev. Biochem. 57:957-989 (1988), and Kaiser, et al, Science 243: 187-198 (1989). The polypeptides are purified so that they are substantially free of chemical precursors or other chemicals using standard peptide purification techniques. The language “substantially free of chemical precursors or other chemicals” includes preparations of peptide in which the peptide is separated from chemical precursors or other chemicals that are involved in the synthesis of the peptide. In one embodiment, the language “substantially free of chemical precursors or other chemicals” includes preparations of peptide having less than about 30% (by dry weight) of chemical precursors or non-peptide chemicals, more preferably less than about 20% chemical precursors or non-peptide chemicals, still more preferably less than about 10% chemical precursors or non-peptide chemicals, and most preferably less than about 5% chemical precursors or non-peptide chemicals.

Chemical synthesis of polypeptides facilitates the incorporation of modified or unnatural amino acids, including D-amino acids and other small organic molecules. Replacement of one or more L-amino acids in a peptide with the corresponding D-amino acid isoforms can be used to increase the resistance of peptides to enzymatic hydrolysis, and to enhance one or more properties of biologically active peptides, i.e., receptor binding, functional potency or duration of action. See, e.g. Doherty, et al., 1993. J. Med. Chem. 36: 2585-2594; Kirby, et al., 1993. J. Med. Chem. 36:3802-3808; Morita, et al., 1994. FEBS Lett. 353: 84-88; Wang, et al., 1993. Int. J. Pept. Protein Res. 42: 392-399; Fauchere and Thiunieau, 1992. Adv. Drug Res. 23: 127-159.

Introduction of covalent cross-links into a peptide sequence can conformationally and topographically constrain the polypeptide backbone. This strategy can be used to develop peptide analogs of the fusion polypeptides with increased potency, selectivity and stability. Because the conformational entropy of a cyclic peptide is lower than its linear counterpart, adoption of a specific conformation may occur with a smaller decrease in entropy for a cyclic analog than for an acyclic analog, thereby making the free energy for binding more favorable. Macrocyclization is often accomplished by forming an amide bond between the peptide N- and C-termini, between a side chain and the N- or C-terminus [e.g., with K₃Fe(CN)₆at pH 8.5] (Samson et al., Endocrinology, 137: 5182-5185 (1996)), or between two amino acid side chains. See, e.g. DeGrado, Adv Protein Chem, 39: 51-124 (1988). Disulfide bridges are also introduced into linear sequences to reduce their flexibility. See, e.g. Rose, et al., Adv Protein Chem, 37: 1-109 (1985); Mosberg et al., Biochem Biophys Res Commun, 106: 505-512 (1982). Furthermore, the replacement of cysteine residues with penicillamine (Pen, 3-mercapto-(D) valine) has been used to increase the selectivity of some opioid-receptor interactions. Lipkowski and Carr, Peptides: Synthesis, Structures, and Applications, Gutte, ed., Academic Press pp. 287-320 (1995).

### Methods of Decreasing Toxin A Binding to a Host Cell

Cell surface binding of Toxin A is inhibited (e.g. decreased) by contacting a cell with the αGal fusion peptide of the invention. The αGal fusion peptide sterically inhibits cell surface binding of the bacterial toxin, thereby preventing bacterial toxin infection. Alternatively, cell surface binding of Toxin A and/or Toxin A producing bacteria is inhibited (e.g., decreased) by contacting Toxin A and/or Toxin A producing bacteria with the αGal fusion peptide of the invention, whereby the αGal fusion peptide binds to Toxin A, thereby preventing Toxin A from binding to its natural epitope, thereby preventing bacterial toxin infection. The Toxin A producing bacteria is, for example, C. difficile.

Inhibition of attachment is characterized by a decrease in cell internalization and thereby decrease in glucosylation of Rho proteins in the cytosol. The αGal fusion peptide is contacted with one or more cells of a subject by systemic and/or rectal administration of the SI fusion peptide to the subject. The αGal fusion peptide is administered in an amount sufficient to decrease (e.g., inhibit) bacterial toxin-cell surface binding and/or internalization. Toxin A and/or C. difficile are directly contacted with the αGal fusion polypeptides of the invention. Alternatively, Toxin A and/or Toxin A producing bacteria is directly contacted with the αGal fusion peptide. Toxin A cell surface binding is measured using standard immunocytochemical assays known in the art, e.g. by measuring toxin binding to cells using radioactively, or by other means, labeled toxins, and/or by detecting attached toxins using anti-Toxin A antibodies.

The methods are useful to alleviate the symptoms of infection by Toxin A producing bacteria or a disease associated with infection by Toxin A producing bacteria. Signs and symptoms associated with infection by Toxin A include for example, exposure to antibiotics, diarrhea, abdominal pain, and foul stool odor.

The methods described herein lead to a reduction in the severity or the alleviation of one or more symptoms of infection by Toxin A produced by C. difficile or disorder such as those described herein. Toxin A infection or disorders associated with infection by Toxin A produced by C. difficile are diagnosed and or monitored, typically by a physician using standard methodologies.

The subject is e.g. any mammal, e.g. a human, a primate, mouse, rat, dog, cat, cow, horse, pig. The treatment is administered prior to bacterial toxin infection or diagnosis of the disorder. Alternatively, treatment is administered after a subject has an infection.

Efficaciousness of treatment is determined in association with any known method for diagnosing or treating the particular bacterial toxin infection or disorder associated with a bacterial toxin infection. Alleviation of one or more symptoms of the bacterial toxin infection or disorder indicates that the compound confers a clinical benefit.

### Pharmaceutical Compositions Including αGal Fusion Polypeptides or Nucleic Acids Encoding Same

The αGal fusion proteins, or nucleic acid molecules encoding these fusion proteins, (also referred to herein as “Therapeutics” or “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The active agents disclosed herein can also be formulated as liposomes. Liposomes are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an αGal fusion protein) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

In some embodiments, oral or parenteral compositions are formulated in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g. U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

Sustained-release preparations can be prepared, if desired. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(-)-3-hydroxybutyric acid. While polymers such as ethylene-vinyl acetate and lactic acid-glycolic acid enable release of molecules for over 100 days, certain hydrogels release proteins for shorter time periods.

The pharmaceutical compositions can be included in a container, pack, or dispenser together with instructions for administration.

### Abbreviations

The following abbreviations are used herein:

ADCC, antibody-dependent cellular cytotoxicity; BSA, bovine serum albumin; DXR, delayed xenorejection; ELISA, enzyme-linked immunosorbent assay; FT, fucosyltransferase; Gal, D-galactose; GT, galactosyltransferase; Glc, D-glucose; GlcNAc, D-N-ERROR; GlyCAM-1, glycosylation-dependent cell adhesion molecule-1; HAR, hyperacute rejection; Ig, immunoglobulin; MAdCAM-1, mucosal addressin cell adhesion molecule; PAEC, porcine aortic endothelial cells; PBMC, peripheral blood mononuclear cells; PSGL-1, P-selectin glycoprotein ligand-1; RBC, red blood cell; SDS-PAGE, sodium dodecyl sulphate—polyacrylamide gel electrophoresis; Hex, hexose; HexNAc, N-acetyl hexosamine; NeuAc, N-acetyl neuraminic acid; NeuGc, N-glycolyl neuraminic acid; and HexNol is the open (not the ring) form of N-acetyl hexosamine.

The invention will be further illustrated in the following non-limiting examples.

### EXAMPLE 1

### Transient Expression of Substituted Recominant P-Selectin Glycoprotein Ligand/Immunoglobulin Fusion Proteins

### General Methods

Cell culture COS-7 m6 cells (35) were passaged in Dulbecco's modified Eagle's medium (DMEM), with 10% fetal bovine serum (FBS) and 25 μg/ml gentamicin sulfate.

Construction of expression vectors

The porcine α 1,3 GT (37-39) was PCR amplified off pig spleen cDNA using a forward primer having six codons of complementarity to the 5′ end of the coding sequence, a Kozak translational initiation concensus sequence and a Hind3 restriction site, and a reverse primer with six codons of complementarity to the 3′ end of the coding sequence, a translational stop and a Not1 restriction site. The amplified α 1,3GT cDNA was cloned into the polylinker of CDM8 using Hind3 and Not1 (35). The P-selectin glycoprotein ligand-1 (PSGL-1) a highly glycosylated mucin-type protein mediating binding to P-selectin (40) coding sequence was obtained by PCR off an HL-60 cDNA library, cloned into CDM8 with Hind3 and Not1, and confirmed by DNA sequencing. The mucin/immunoglobulin expression plasmid was constructed by fusing the PCR-amplified cDNA of the extracellular part of PSGL-1 in frame via a BamH1 site, to the Fc part (hinge, CH2 and CH3) of mouse IgG_{2b} carried as an expression casette in CDM7 (Seed, B. et al).

Production and purification of secreted mucin/immunoglobulin chimeras

COS m6 cell were transfected using the DEAE-dextran protocol and 1 μg of CsCl-gradient purified plasmid DNA per ml transfection cocktail. COS cells were transfected at approximately 70% confluency with empty vector (CDM8), the PSGL1/mIgG_{2b} plasmid alone or in combination with the α 1,3GT encoding plasmid. Transfected cells were trypsinized and transferred to new flasks the day after transfection. Following adherence for approximately 12 hrs, the medium was discarded, the cells washed with phosphate buffered saline (PBS), and subsequently incubated another 7 days in serum-free, AIM-V medium (cat.nr. 12030, Life technologies Inc.). After incubation, supernatants were collected, debris spun down (1400×g, 20 minutes), and NaN.sub.3 added to 0.02%. PSGL1/mIgG.sub.2b fusion protein was purified on goat anti-mouse IgG agarose beads (A-6531, Sigma) by rolling head over tail, over night at 4.degree. C. The beads were washed in PBS and subsequently used for SDS-PAGE and Western blot analysis, or for absorption of human AB serum and purified human immunoglobulins.

Purification of human IgG, IgM and IgA

Human IgG, IgM and IgA were purified from human AB serum—pooled from more than 20 healthy blood donors—using goat anti-human IgG (Fc specific; A-3316, Sigma), IgM (μ-chain specific; A-9935, Sigma), and IgA (α-chain specific; A-2691, Sigma) agarose beads. Briefly, 5 ml of slurry (2.5 ml packed beads) were poured into a column of 10 mm diameter and washed with PBS. Ten milliter of human pooled AB serum was applied at 1 ml/minute using a peristaltic pump, washed with several column volumns of PBS, and eluted with 0.1M glycine, 0. 15M NaCl, pH 2.4 using a flow rate of 1 ml/minute. One milliliter fractions were collected in tubes containing 0.7 ml of neutralizing buffer (0.2M Tris/HCl, pH 9). The absorption at 280 nm was read spectrophotometrically and tubes containing protein were pooled. dialyzed against 1% PBS, and lyophilized. Lyophilized immunoglobulins were resuspended in distilled water and the concentrations adjusted to 16 mg/ml for IgG, 4 mg/ml for IgA and 2 mg/ml for IgM.

### SDS-PAGE and Western Blotting

SDS-PAGE was run by the method of Leammli with a 5% stacking gel and a 6 or 10% resolving gel using a vertical Mini-PROTEAN II electrophoresis system (Bio-Rad, Herculus, Calif.) (41). Separated proteins were electrophoretically blotted onto Hybond.TM.-C extra membranes (Amersham) using a Mini Trans-Blot electrophoretic transfer cell (Bio-Rad, Herculus, Calif.) (42). Protein gels were stained using a silver staining kit according to the manufacturer's instructions (Bio-Rad, Herculus, Calif.). Following blocking for at least 2 hrs in 3% BSA in PBS, the membranes were probed for 2 hrs in room temperature with peroxidase-conjugated Bandereia simplicifolia isolectin B.sub.4 (L-5391, Sigma) diluted to a concentration of 1 μg/ml in PBS, pH 6.8 containing 0.2 mM CaCl₂. The membranes were washed 5 times with PBS, pH 6.8, and bound lectin was visualized by chemiluminescens using the ECL.TM. kit according to the instructions of the manufacturer (Amersham).

Quantification of PSGLb1/mIgG_{2b} by Anti-Mouse IgG Fc ELISA

The concentration of fusion protein in cell culture supernatants before and after absorption was determined by a 96-well ELISA assay, in which fusion proteins were captured with an affinity purified, polyclonal goat anti-mouse IgG Fc antibody (cat.nr. 55482, Cappel/Organon Teknika, Durham, N.C.). Following blocking with 3% BSA in PBS, the fusion proteins were captured and detected with a peroxidase-conjugated, affinity purified, polyclonal anti-mouse IgG Fc antibody (cat.nr. 55566, Organon Teknika, Durham, N.C.) using O-phenylenediamine dihydrochloride as substrate (Sigma). The plate was read at 492 nm and the ELISA calibrated using a dilution series of purified mouse IgG Fc fragments (cat.nr. 015-000-008, Jackson ImmunoResearch Labs, Inc., West Grove, Pa.) resuspended in AIM V serum-free medium.

### Results

Expression and Characterization of the PSGL1/mIgG_{2b} Fusion Protein

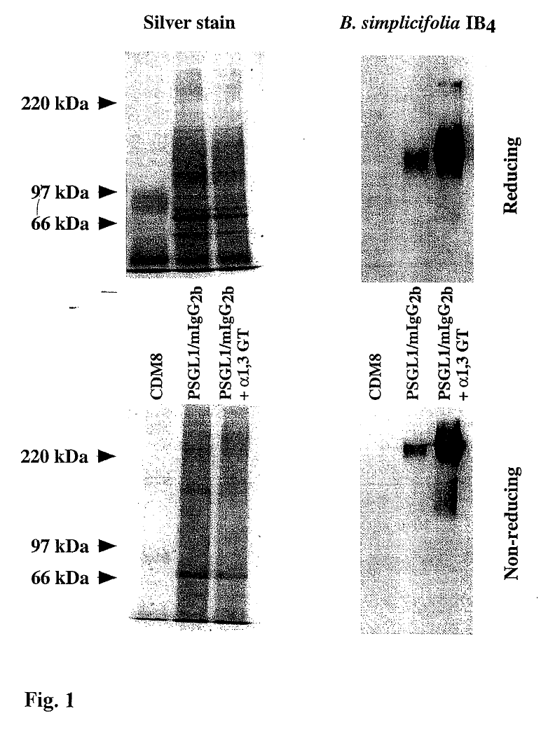

Supernatants from COS-7 m6 cells transfected with the vector plasmid CDM8, the PSGL1/mIgG_{2b} plasmid, or the, PSGL1/mIgG_{2b} together with the porcine α1,3 GT plasmid, were collected approximately 7 days after transfection. Secreted mucin/Ig fusion proteins were purified by absorption on anti-mouse IgG agarose beads and subjected to SDS-PAGE and Western blotting using the Bandereia simplicifolia isolectin B₄ (BSA IB₄) for detection. As seen in FIG. 1, the fusion protein migrated under reducing conditions as a broad band with an apparent molecular weight of 145 kDa that stained relatively poorly with silver. The heterogeneity in size, approximately 125 to 165 kDa, and poor stainability is in concordance with previous observations with respect to the behavior of highly glycosylated, mucin-type proteins (43, 44). The fusion protein is most likely produced as a homodimer because SDS-PAGE under non-reducing conditions revealed a double-band of an apparent molecular weight of more than 250 kDa. The amounts of fusion protein affinity-purified from the two supernatants derived from the same number of COS cells transfected with the PSGL1/mIgG_{2b} plasmid alone or together with the α1,3GT plasmid, respectively, were similar. Probing the electroblotted membranes with BSA IB.sub.4 revealed strong staining of the fusion protein obtained following cotransfection with the porcine α1,3 GT (FIG. 1). It is clear, though, that the PSGL1/mIgG_{2b} fusion protein produced in COS-7 m6 cells without cotransfection of the α1,3 GT CDNA also exhibited weak staining with the BSA IB₄ lectin, in spite of the fact that COS cells are derived from the Simian monkey—an old world monkey lacking α1,3 GT activity. This indicates that the BSA IB₄ lectin has a slightly broader specificity than just Galα1,3Gal epitopes (45). Nevertheless, cotransfection of the porcine α1,3GT cDNA supported the expression of a highly Galα1,3Gal-substituted PSGL1/mIgG_{2b} fusion protein.

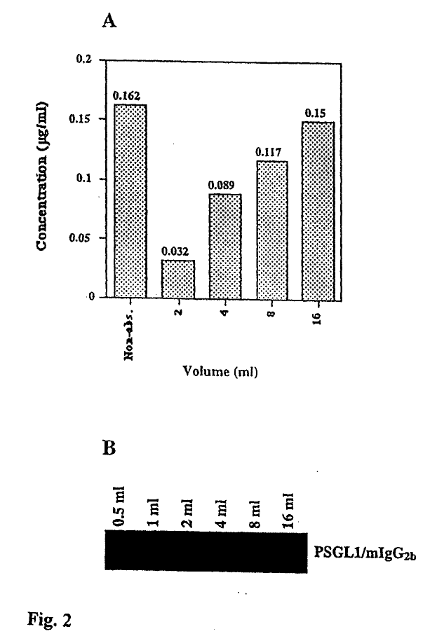

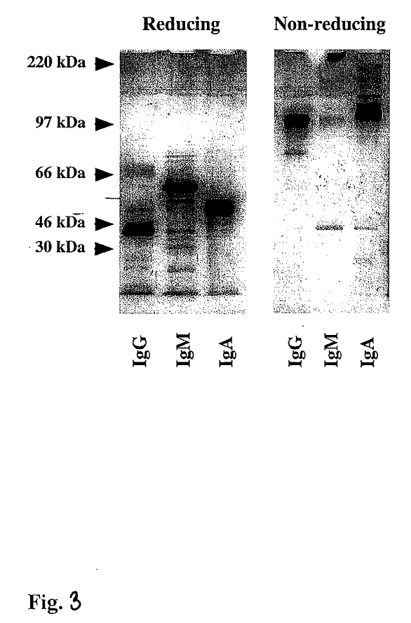

Quantification of PSGL1/mIgG_{2b} chimeras in supernatants of transfected COS cells, and on goat anti-mouse IgG agarose beads following absorption. A sandwich ELISA was employed to quantify the amount of PSGL1/mIgG_{2b} in the supernatants of transfected COS cells. Typically, 5 culture flasks (260 ml flasks, Nunclon.TM.) with COS cells at 70% confluence were transfected and incubated as described in materials and methods. Following an incubation period of 7 days in 10 ml of AIM V medium per flask, the medium was collected. The concentration of fusion protein in the supernatant from such a transfection, as well as in different volumes of supernatant following absorption on 100 μl gel slurry of anti-mouse IgG agarose beads (corresponding to 50 μl packed beads) was determined by an ELISA calibrated with purified mouse IgG Fc fragments (FIG. 2). The concentration of PSGL1/mIgG_{2b} in the supernatants ranged from 150 to 200 ng/μl, and in this particular experiment it was approximately 160 ng/μl (FIG. 2A, the non-absorbed column). The concentration of PSGL1/mIgG_{2b} remaining in 2, 4 and 8 ml of supernatant following absorption on 50 μl packed anti-mouse IgG agarose beads was 32, 89 and 117 ng/μl, respectively. This corresponds to 260, 290 and 360 ng of PSGL1/mIgG_{2b} being absorbed onto 50 μl packed anti-mouse IgG agarose beads from 2, 4 and 8 ml of supernatant, respectively. Western blot analysis with the B. simplicifolia IB₄ lectin revealed that 50 μl of packed beads could absorb out the PSGL1/mIgG_{2b} fusion protein from 1 ml supernatant to below detectability and from 2 ml to barely detectable levels (not shown).

### EXAMPLE 2

### Stable Expression of Substituted Recominant P-Selectin Glycoprotein Ligand/Immunoglobulin Fusion Proteins

### General Methods Cell Culture

CHO-K1, COS7m6, and 293T cells were cultured in Dulbecco's modified Eagle's medium (DMEM) with 10% fetal bovine serum (FBS) and 25 μg/ml gentamicin sulfate. The selection media contained puromycin (cat. no. P7255; Sigma, St. Louis, Mo. 63178), hygromycin (cat. no. 400051; Calbiochem, La Jolla, Calif. 92039), and G418 (cat. no. G7034; Sigma, St. Louis, Mo. 63178) as indicated below.

### Construction of Expression Plasmids

The porcine α1,3GalT (Gustafsson, K. et al., 1994) and PSGL-1/mIgG_{2b} expression plasmids were constructed as described (Liu, J. et al., 1997). The C2 GnTI cDNA was amplified by PCR from an HL60 cDNA library using cgcgggctcgagatgaagatattcaaatgt (SEQ ID NO: 2) and cgcggggcggccgctcatgatgtggtagtgagat (SEQ ID NO: 3) as forward and reverse primers, respectively. The vectors used to generate stable transfectants were bidirectional having the EF1α promoter upstream of a polylinker, a splice donor and acceptor site, and the bidirectional poly(A) addition signal of SV40; opposite in orientation to this transcription unit, and utilizing the poly(A) signals from the opposite direction was a second transcription unit consisting of the HSV TK promoter followed by the coding sequences for puromycin acetyltransferase (EF1α/PAC), the hygromycin b (EF 1α/Hyg), and the neomycin (EFF1α/Neo) resistance genes (N. Chiu, J. Holgersson and B. Seed). The cDNAs of porcine α1,3GalT and PSGL-1/mIgG_{2b} were swapped into the EF1α/Hyg and EF 1α/PAC vectors, respectively, using Hind III and Not I. The gene of C2GnTI was swapped into EF1 α/Neo using Xho I and Not I.

### DNA Transfection and Clonal Selection

Adherent CHO-K1, COS7m6 and 293T cells were seeded in 75 cm² T-flasks and were transfected approximately 24 hours later at a cell confluency of 70-80%. A modified polyethylenimine (PEI) transfection method was used for transfection (Boussif, O. et al., 1995; He, Z. et al., 2001). Twenty-four hours after transfection, cells in each T-flask were split into five 100 mm petri dishes and incubated in selection medium. The concentration of puromycin in the selection medium was 6.0, 1.5, and 1.0 μg/ml respectively, for CHO-K1, COS7m6 and 293T cells. A hygromycin b concentration of 550, 50, and 100 μg/ml was used for CHO-K1, COS7m6 and 293T cells, respectively, and a G418 concentration of 900 μg/ml was used for CHO-K1 cells. The selection medium was changed every third day. The drug resistant clones formed after approximately two weeks. Clones were identified under the microscope and hand-picked using a pipetman. Selected colonies were cultured in 96-well plates in the presence of selection drugs for another two weeks. Cell culture supernatants were harvested when the cells had reached 80-90% confluency, and the concentration of PSGL-1/mIgG_{2b} was assessed by ELISA, SDS-PAGE and Western blotting using a goat anti-mouse IgG Fc antibody. The CHO-K1, COS7m6 and 293T clones with the highest PSGL-1/mIgG_{2b} expression were transfected with the porcine α1,3GalT encoding plasmid and selected in hygromycin-containing medium. Resistant clones were isolated and characterized by ELISA, SDS-PAGE and Western blot using both a goat anti-mouse IgG Fc antibody and the GSA I IB₄-lectin recognizing terminal α-Gal. Two CHO clones with a high relative α-Gal expression on PSGL-1/mIgG_{2b} were further transfected with the C2 GnTI and selected in G418-containing medium. Expression of C2 GnTI was verified by an increase in size of PSGL-1/mIgG_{2b} indicating more complex O-glycans.

### SDS-PAGE and Western Blotting

SDS-PAGE was run by the method of Laemmli (Laemmli, U. K., 1970) with 5% stacking gels and 8% resolving gels using a vertical Mini-Protean II electrophoresis system (Bio-Rad, Hercules, Calif., USA). Samples were electrophoretically run under reducing and non-reducing conditions. In order to increase the resolution, 4-15% gradient gels (cat.no. 161-1104; Bio-Rad, Hercules, Calif., USA), or 4-12% gradient gels (cat.no NP0322; Invitrogen, Lidingö, Sweden) were used in some experiments. The latter gels were used in combination with the MES buffer (cat.noNP0002; Invitrogen). A precision protein standard (cat.no RPN756; Amersham Biosciences, Uppsala, Sweden) was applied as a reference for protein molecular weight determination. Protein gels were stained using the Pro Q Emerald 300 Glycoprotein detection kit in combination with Ruby (cat.no P21855; Molecular Probes, Leiden, The Netherlands). These gels were visualized in a Flour-S Max MultiImager carrying a CCD camera. Separated proteins were also electrophoretically blotted onto Hybond C extra membranes (cat.no. RPN203E; Amersham Biosciences), or nitrocellulose membranes (cat.no LC2001; Invitrogen) using a Mini TransBlot (Bio-Rad) electrophoretic transfer cell (Towbin, H. et al., 1979). Following blocking for 1 hour in 3% BSA in PBS with 0.2% Tween 20, the membranes were probed for one hour at room temperature with peroxidase-conjugated GSA I IB₄-lectin (cat.no. L-5391; Sigma) diluted to a concentration of 1 μg/ml, peroxidase-conjugated goat anti-mouse IgG Fc antibodies (cat.no. A-9917; Sigma) diluted 1:1,000, and a mouse anti-PSGL-1 antibody (clone KPL-1, cat.no 557502; BD PharMingen, San Diego, Calif.) diluted 1:1,000. Secondary antibody was a peroxidase-conjugated goat anti-mouse IgG F(ab)′₂ (cat.no A 2304; Sigma) diluted 1:50,000. All dilutions were done in blocking buffer. The membranes were washed three times with PBS containing 0.2% Tween 20 between and after incubations. Bound lectins and antibodies were visualized by chemiluminescence using the ECL kit according to the manufacturer's instructions (cat.no. RPN 2106; Amersham Biosciences).

α-Gal Epitope Density on, and Quantification of, PSGL-1/mIgG_{2b} Using an Enzyme-Linked Immunosorbent Assay

The concentration of recombinant PSGL-1/mIgG_{2b} in cell culture supernatants, and its relative α-Gal epitope density, was determined by a two-antibody sandwich ELISA. The 96-well ELISA plate was coated overnight at 4° C. with an affinity-purified, polyclonal goat anti-mouse IgG Fc antibody (cat. nr. 55482; Cappel/Organon Teknika, Durham, N.C.) at a concentration of 20 μg/ml. The plate was blocked with 1% BSA in PBS for 1 hour. The supernatant containing PSGL-1/mIgG_{2b} was incubated for 4 hours and then washed three times with PBS containing 0.5% (v/v) Tween 20. After washing, the plate was incubated with a peroxidase-conjugated, anti-mouse IgG Fc antibody (cat.no. A-9917; Sigma) in a 1:3,000 dilution or with peroxidase-conjugated GSA I IB₄-lectin (cat.no. L-5391 ;Sigma) diluted 1:2,000, for two hours. Bound peroxidase-conjugated antibody or peroxidase-conjugated GSA-lectin was visualized with 3,3′,5,5′-Tetramethylbenzidine dihydrochloride (cat. nr. T-3405; Sigma, Sweden). The reaction was stopped by 2M H₂SO₄ and the plates read at 450 nm. The PSGL-1/mIgG_{2b} concentration was estimated using for calibration a dilution series of purified mouse IgG Fc fragments (cat. Nr. 015-000-008; Jackson ImmunoResearch Labs., Inc., West Grove, Pa.) resuspended in the medium used for fusion protein production or in PBS containing 1% BSA. The α-Gal epitope density was determined by comparing the relative O.D. from the two ELISAs (GSA-reactivity/anti-mouse IgG reactivity).

### Stirred Flask Batch Cultures of CHO Clones

Each batch culture was started with 6.0×10⁷ cells (representing ten 175cm ² T-flasks with cells of 90-100 % confluency). After digestion with trypsin (0.5 mg/ml)-EDTA (0.2 mg/ml), cells were resuspended in a small volume of medium and centrifuged at 200×g for 5 minutes to remove excess of trypsin. The cell density was determined by counting the cells in a Bürker chamber, and medium was added to a final concentration of 3.0×10⁵ cells/ml. The cell suspension was transferred to IL stirred flasks and a cell spin device (Integra Biosciences, Wallisellen, Switzerland) was utilized in order to stir the cultures at a speed of 60 rpm. PSGL-1/mIgG_{2b} secreting CHO-K1 cells expressing α1,3GalT alone or in combination with C2 GnTI, were cultured in the presence of puromycin (200 μg/ml), or puromycin (200 μg/ml) and G418 (500 μg/ml), respectively. The cells were counted every second day. When the cell density reached 5.0×10⁵ cells/ml, new medium was added so that the cell density once again equalled 3.0×10⁵ cells/ml. This was repeated until the cell suspension volume reached 1,000 ml. Cells were then continuously cultured until cell viability was reduced to 50%.

Purification of Recombinant PSGL-1/mIgG_{2b}

The supernatants were cleared from debris by centrifugation at 1,420×g for 20 minutes. Cleared supernatants were passed through a column containing 10 ml of goat anti-mouse IgG (whole molecule)-agarose (cat.no. A 6531; Sigma) at a flow rate of 0.5 ml/min. Following washing with 120 ml of PBS, bound fusion protein was eluted with 120 ml of 3 M NaSCN. The contents of the tubes containing the fusion protein was pooled following analysis by SDS-PAGE and Western blotting using anti-mouse IgG for detection. The fraction with PSGL-1/mIgG_{2b} was dialyzed against distilled water, lyophilised, and resuspended in 1-2 ml of distilled H₂O. The concentration of the fusion protein was determined by ELISA. To remove low molecular weight contaminants, the fusion protein was further purified by gel filtration on a HiPrep 16/60 Sephacryl S-200 HR column (cat.no. 17-1166-01; Amersham Biosciences, Uppsala, Sweden) eluted with PBS at a flow rate of 0.5 ml/min using a FPLC (Pharmacia Biotech, Sweden). Five-ml fractions were collected and tubes containing protein were identified by UV spectrophotometry at 280 nm. Pooled fractions were again analyzed by SDS-PAGE and Western blotting, pooled, dialyzed and resuspended in distilled water.

Chemical Release and Permethylation of O-Linked Glycans from Purified PSGL-1/mIgG_{2b}

Oligosaccharides were released by β-elimination as described (Carlstedt, I. et al., 1993). Released oligosaccharides were evaporated under a stream of nitrogen at 45° C., and permethylated according to Ciucanu and Kerek (Ciucanu, I. et al., 1984), with slight modifications as described (Hansson, G. C. et al., 1993).

### Mass Spectrometry

Electrospray ionization-mass spectrometry (ESI-MS) in positive-ion mode was performed using an LCQ ion-trap mass spectrometer (ThermoFinnigan, San Jose, Calif.). The sample was dissolved in methanol:water (1:1) and introduced into the mass spectrometer at a flow rate of 5-10 μl/min. Nitrogen was used as sheath gas and the needle voltage set to 4.0 kV. The temperature of the heated capillary was set to 200° C. A total of 10-20 spectra were summed to yield the ESI-MS and ESI-MS/MS spectra.

### Results

Stable Expression of α-Gal Substituted P-selectin Glycoprotein ligand-1/Mouse IgG_{2b} in Different Host Cells

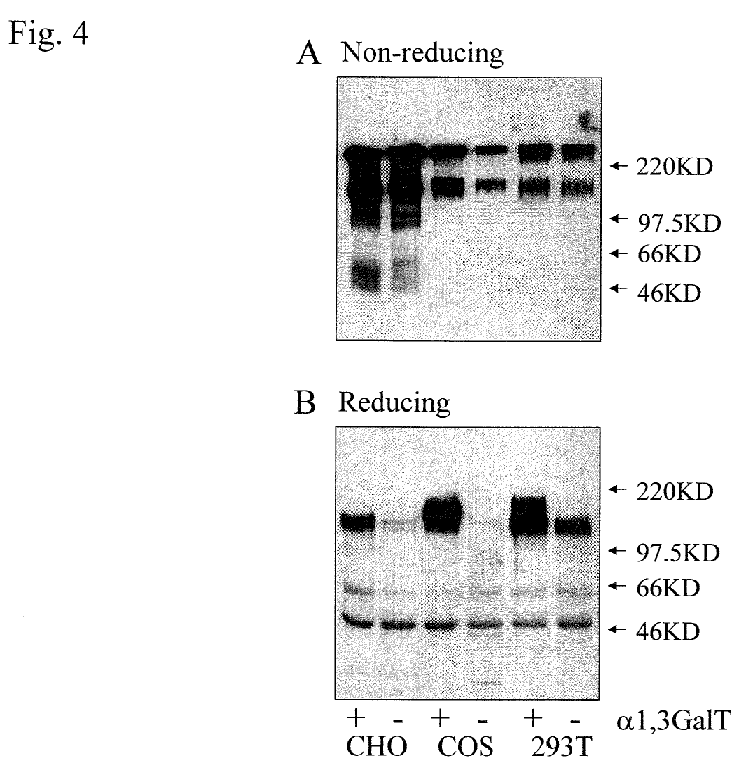

Following 15-20 days of culture in selection medium supplemented with puromycin, differently sized colonies of CHO-K1, COS7m6 and 293T cells were identified by phase contrast microscopy. Under the microscope, 192 colonies of each cell type were picked by pipette and transferred to two 96-well plates for further expansion under selection. An Ig sandwich ELISA was used to assess fusion protein concentration in the supernatants of individual clones, and 31 CHO-K1, 8 COS7m6 and 36 293T colonies were anti-mouse IgG Fc positive. The top five secreting colonies from each cell line were moved to a 24-well plate and further expanded. The best expressing CHO-K1, COS7m6 and 293T clones were transfected with the α1,3GalT-encoding plasmid carrying the hygromycin B resistance gene. PSGL-1/mIgG₂b expressing cells that had stably integrated the α1,3GalT gene were selected using both puromycin and hygromycin. Twenty-seven CHO-K1, 3 COS7m6 and 31 293T colonies were selected. Colonies to be expanded were chosen based on the concentration of fusion protein and its relative level of α-Gal epitope substitution as determined in anti-mouse IgG and Griffonia simplicifolia I IB₄ lectin ELISAs. Immuno-affinity isolated PSGL-1/mIgG_{2b} expressed in CHO-K1 (clone 5L4-1 and 10, respectively), COS7m6 (clone 5I and 2H5, respectively) and 293T (clone 14 and C, respectively) cells with or without the porcine α1,3GalT was characterized by SDS-PAGE and Western blotting (Table I, FIG. 4). All cell lines produced an anti-mouse IgG Fc-reactive protein of approximately 300 kDa under non-reducing conditions (FIG. 4A). In accordance with previous observations (Liu, J. et al., 1997; Liu, J. et al., 2003), PSGL-1/mIgG_{2b} was produced as a dimer as indicated by the reduction to half the size upon reduction (compare FIG. 4A and B). The presence of α-Gal epitopes on the fusion protein made in the different cell types was detected using the GSA I IB₄ lectin (FIG. 4B). Co-expression of the α1,3GalT in CHO-K1 (clone 5L4-1), COS7m6 (clone 5I) and 293T (clone 14) led to expression of α-Gal epitopes on the fusion protein as detected by the lectin. The lectin reactivity of PSGL-1/mIgG_{2b} made in 293T cells without the α1,3GalT was unexpected, and indicates the presence of α-Gal residues other than the Galili antigen on that fusion protein (FIG. 4B). The fusion protein produced in COS and 293T cells in the presence of α1,3GalT contained glycoforms of bigger size than the fusion protein produced in CHO-K1 cells (FIG. 4B).

TABLE 1
CHO, COS and 293T derived cell clones

| Cell clone | PSGL-1/mIgG_{2b} | α1,3GalT | C2 GnTI |
|---|---|---|---|
| CHO-10 | X | | |
| CHO-5L4-1 | X | X | |
| CHO-C2-1-9 | X | X | X |
| COS-2H5 | X | | |
| COS-5I | X | X | |
| 293T-C | X | | |
| 293T-14 | X | X | |

The α-Gal Epitope Density on PSGL-1/mIgG_{2b} is Dependent on the Host Cell Used for Its Production The relative α-Gal epitope density on PSGL-1/mIgG_{2b} made in CHO, COS and 293T cells was determined by ELISA (FIG. 8). PSGL-1/mIgG_{2b} made in COS cells in the presence of the α1,3GalT exhibited a 5.3-fold increase in the relative O.D. (GSA-reactivity/anti-mouse IgG reactivity) compared to PSGL-1/mIgG_{2b} made in COS without the α1,3GalT (FIG. 5). For 293T cells there was a 3.1-fold increase in the relative O.D., and for CHO cells there was just a 1.8-fold increase (FIG. 5). The ELISA results were in agreement with the relative GSA lectin staining seen in the Western blot experiments of immuno-affinity purified PSGL-1/mIgG_{2b} (FIG. 4B).
Co-Expression of a Core 2 β1,6 GlcNAc Transferase in CHO Cells Improves PSGL-1/mIgG_{2b} α-Gal Epitope Density

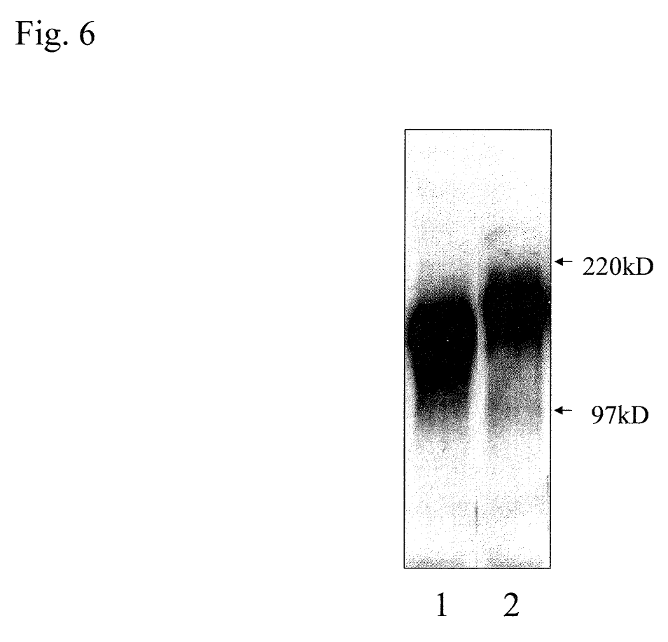

In an attempt to increase the number of α-Gal epitopes on CHO-K1 cell-secreted mucin/Igs, CHO-K1 cells stably expressing PSGL-1/mIgG_{2b}, α1,3GalT and C2 GnTI were established, and PSGL-1/mIgG_{2b} secreted by those cells were analyzed by ELISA, SDS-PAGE and Western blot using the anti-mouse IgG antibody and GSA I IB₄. The apparent MW of PSGL-1/mIgG_{2b} increased following stable expression of the core 2 enzyme indicating more complex glycans on the fusion protein (FIG. 6). The α-Gal epitope density on PSGL-1/mIgG_{2b} showed a 13.0-fold increase compared to PSGL-1/mIgG_{2b} made in CHO-K1 cells without the α1,3GalT and a 7.4-fold increase compared to PSGL-1/mIgG_{2b} made with the α1,3GalT alone (FIG. 5).

Purification of Recombinant PSGL-1/mIgG_{2b} for Structural Characterization of its O-Linked Glycans

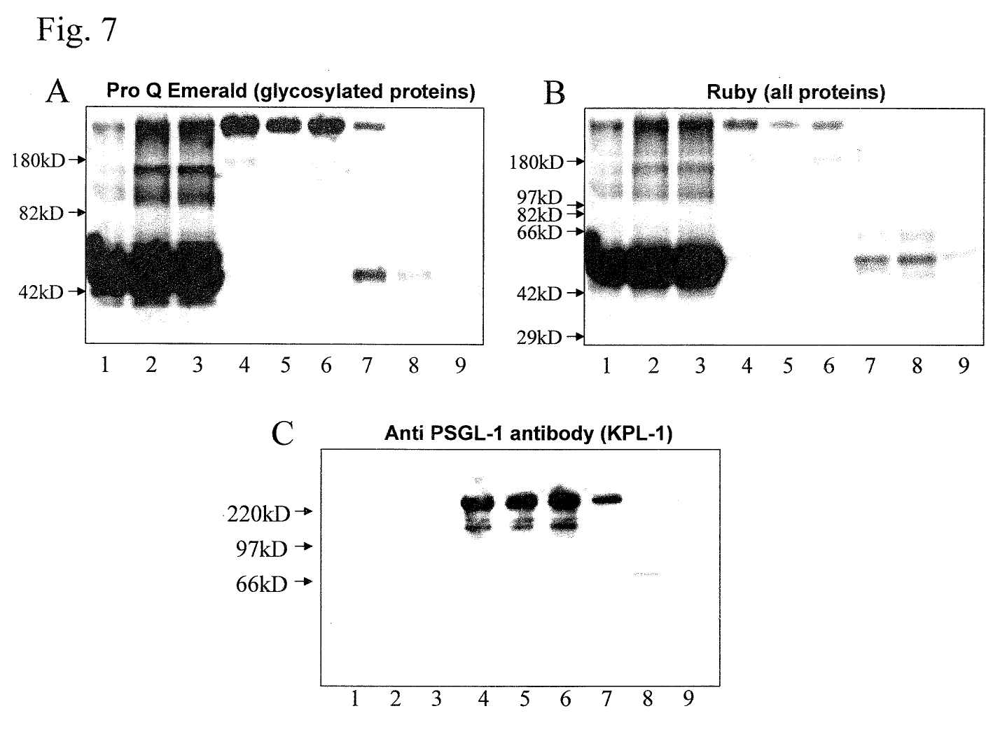

Recombinant PSGL-1/mIgG_{2b} was purified from 1 L stirred flask cultures of stably transfected CHO-K1 cells expressing PSGL-1/mIgG_{2b} alone (clone 10), in combination with the porcine α1,3 GalT (clone 5L4-1) or in combination with the α1,3 GalT and the C2 GnTI (clone C2-1-9). A two-step purification process, involving anti-mouse IgG affinity chromatography and gel filtration, was set up in order to fully remove contaminating glycosylated proteins that could interfere with the O-glycan structural analysis. Affinity purification of two litres of cell supernatant from each cell clone resulted in 2.2 mg, 1.2 mg and 0.95 mg of PSGL-1/mIgG_{2b} from CHO-10, 5L4-1 and C2-1-9, respectively, as assessed by ELISA. Further purification on a gel filtration column resulted in a final PSGL-1/mIgG_{2b} yield of 0.22 mg, 0. 19 mg and 0.29 mg, respectively. The fractions eluted from the affinity and gel filtration columns were analysed by SDS-PAGE and Western blotting (shown here for clone 10). A glycoprotein staining kit was used in combination with Ruby to detect glycosylated as well as non-glycosylated proteins (FIG. 7A and B), and an anti PSGL-1 antibody confirmed the presence of PSGL-1/mIgG_{2b} (FIG. 7C). This antibody bound strongly to a band of around 300 kDa (FIG. 7C lanes 2 and 4-9) representing the PSGL-1/mIgG_{2b} dimer. A band of around 150 kDa is also seen (lanes 4-6), derived from the fusion protein in its reduced form, as well as a weak band of 60-70 kDa (lanes 7-9) most likely representing fusion protein break down products. In FIG. 11A and B, a 300 kDa band not stained by the anti PSGL-1 antibody can be seen also in lanes 1 and 3, most likely representing a protein derived from the cell culture medium. This is supported also by its presence in the affinity-purified supernatant (lane 3), which indicates that it is not adsorbed on the anti-Ig affinity column. However, a glycosylated band with a MW of 50-60 kDa, not stained by the anti PSGL-1 antibody, can be seen in the affinity purified fraction (FIG. 7A lane 4). This protein is probably also derived from the cell culture medium, and is adsorbed on the affinity column together with the fusion protein. This protein was removed by gel filtration, during which it eluted later (FIG. 7A and B, lanes 7-9) than the fusion protein (FIG. 7A, B and C, lanes 5-6). Additional non-glycosylated proteins of around 50-70 kDa were removed by gel filtration (FIG. 7B, compare lane 4 with lanes 7-9). For each clone, the gel filtration fraction containing the highest amount of fusion protein was chosen for oligosaccharide release. As shown for clone 10 (FIG. 7A and B, lane 5), this fraction did not contain any significant amounts of contaminating proteins, glycosylated or non-glycosylated.

Mass Spectrometry of Permethylated Oligosaccharides Released from Purified, Recombinant PSGL-1/mIgG_{2b}

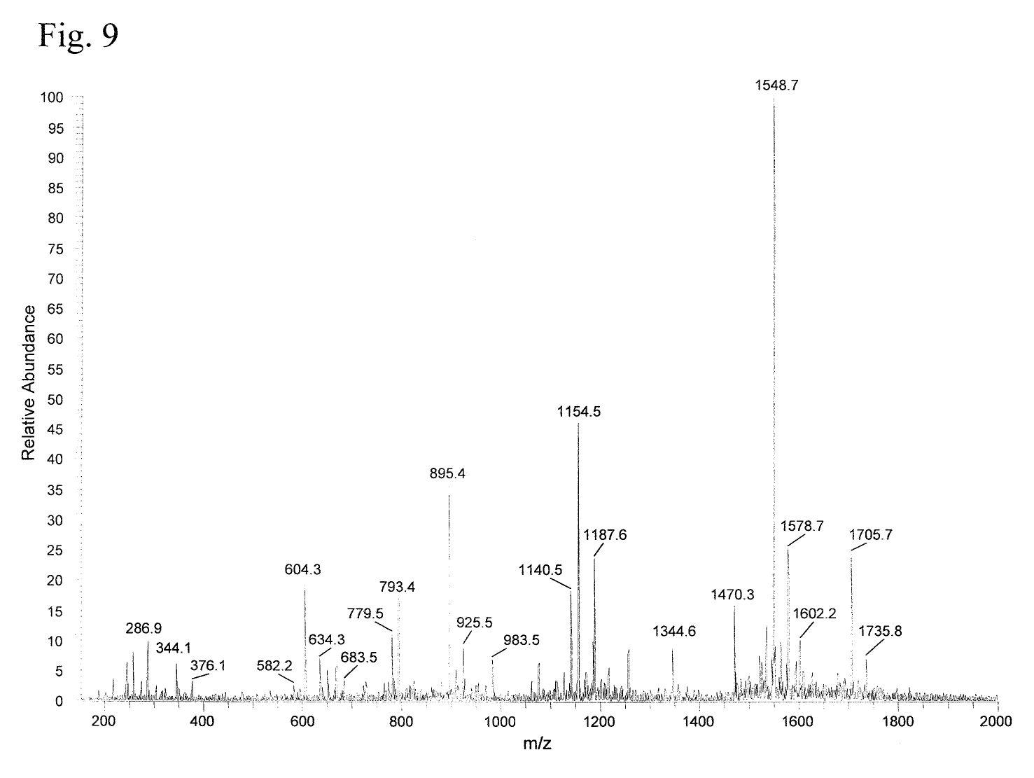

The permethylated oligosaccharides released from clones CHO-10 and 5L4-1 gave similar MS spectra with two predominant groups of peaks around m/z 895.4 and 1256.5 (FIG. 8), while the mass spectrum of O-glycans released from PSGL-1/mIgG_{2b} produced by clone C2-1-9 showed a more complex pattern (FIG. 9). The oligosaccharide sequences of the ions in the ESI-MS spectra were deduced by tandem mass spectrometry (MS/MS). The sequences and tentative structures thus obtained are shown in Table 2. Below, we will describe the results of the MS/MS analyses of the O-glycans on PSGL-1/mIgG_{2b} produced in CHO cells also expressing α1,3GalT and C2 GnTI (C2-1-9). All ions in the MS and MS/MS spectra were detected as sodiated ions.

MS/MS analyses of C2-1-9.

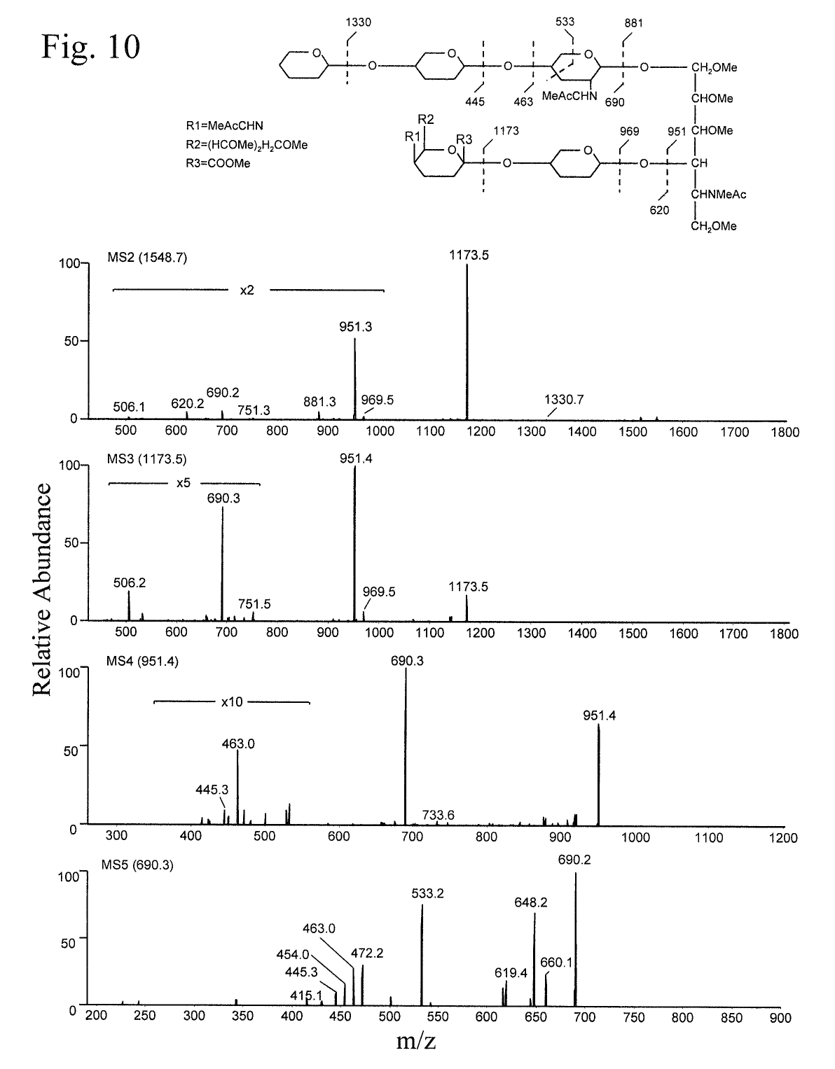

The most intense peak in the mother spectra is a pseudomolecular ion ([M+Na]⁺) at m/z 1548.7 representing a NeuAc-Hex-HexNol-HexN-Hex-Hex structure as assessed by MS/MS in sequential steps (FIG. 10). MS² of this ion gave two major fragment ions at m/z 951.3 ([M—NeuAc-Hex-O+Na]⁺) and 1173.5 ([M—NeuAc +Na]⁺) and several minor at m/z 506.1 ([M—Hex-Hex-HexN—NeuAc+Na]⁺), 620.2 ([NeuAc-Hex-O+Na]⁺), 690.2 ([Hex-Hex-HexN+Na]⁺), 751.3 ([M—Hex-Hex—NeuAc+Na]⁺ or [M—Hex-NeuAc-Hex+Na]⁺), 881.3 ([M—Hex-Hex-HexN+Na]⁺), 969.5 ([M—NeuAc-Hex+Na]⁺) and 1330.7 ([M—Hex+Na]⁺). The fragment ion at m/z 1173.5 was isolated and analyzed by MS³ resulting in fragment ions at m/z 951.4, 506.2, 690.3 and 751.5. The major peak, 951.4, was further analyzed by MS⁴ and gave rise to fragment ions at m/z 445.3 ([Hex-Hex+Na]⁺), 463.0 ([Hex-Hex-O+Na]⁺), 690.3 and 733.6 ([M—Hex—NeuAc-Hex-O+Na]⁺). Finally, the dominant fragment ion in the MS⁴ analysis (690.3) was analyzed by MS⁵. This resulted in sequence ions at m/z 415.1 and 445.3 representing a terminal Hex-Hex, the former ion having lost one oxygen and its methyl group. A Hex-Hex-O structure was also found (463.0). Further, internal Hex-HexN structures were seen, with (472.2) and without (454.0) one oxygen linked to the hexose. Losses of O-Me (660. 1), C-O-Me (648.2) and N-C-O-Me (619.4) from the Hex-Hex-HexN structure was also seen, where the last one probably represents loss of the N-acetyl group from the internal HexN. A major fragment ion at m/z 533.2 was also seen in the MS⁵ spectra. This ion corresponds to a cross-ring fragment of the innermost HexN (FIG. 10), and indicates that the hexose is linked to the HexN in a 1-4 linkage. This sequence is most likely consistent with a sialidated core 2 elongated with a type 2 structure and a terminal Gal.

Apart from the ion at m/z 1548.7, two other pseudomolecular ions possibly terminating with Galα1,3Gal was found in the ESI-MS spectra of clone C2-1-9, at m/z 1578.7 and 1187.6. The ion at m/z 1578.7 was isolated for MS² analysis. The result indicates a NeuGc-Hex-HexNol-HexN-Hex-Hex structure, with fragment ions at m/z 1173.5 ([M−NeuGc+Na]⁺), 951.5 ([M−NeuGc−Hex−O+Na]⁺), 911.3 ([M-Hex-Hex-HexN+Na]⁺), 690.3 ([Hex−Hex−HexN+Na]⁺) and 676.3 ([Hex−Hex−HexN−Me+Na]⁺). MS³ analysis of the 1173.5 ion resulted in a major fragment ion at m/z 951.5 and several minor at m/z 506.1 ([M—Hex-Hex-HexN—NeuAc+Na]⁺), 690.2 and 969.3 ([M -NeuAc-Hex+Na]⁺). The fragment ion at m/z 951.5 was analyzed by MS/MS in a fourth step, giving one major fragment ion at m/z 690.4 and a minor one at m/z 658.2 (690.4-O-Me). However, in the MS² spectra of the ion at m/z 1578.7, unidentified fragment ions at m/z 981.5 and 1203.4 were observed. MS³ and MS⁴ analyses of the ion at m/z 1203.4 resulted in fragment ions at m/z 981.4, 720.4, 690.1, 506.1 and 688.3 (720.1-O-Me). The ion at m/z 720.4, seen in both the MS³ and MS⁴ spectra, is 30 mass units more than the characteristic fragment ion at m/z 690. 1, representing a Hex-Hex-HexN sequence. Unfortunately, further MS/MS analysis of the ion at m/z 720.4 was not possible. The other pseudomolecular ion in the ESI-MS spectra (FIG. 9) with a possible terminal Galα1-3Gal was observed at m/z 1187.6. MS² experiment of this ion resulted in fragment ions at m/z 969.5 ([M—Hex+Na]⁺), 951.4 ([M—Hex-O+Na]⁺), 756.2 ([M-Hex-Hex+Na]⁺), 690.3 ([Hex-Hex-HexN +Na]+), 520.3 ([M—Hex-Hex-HexN+Na]⁺) and 445.1 ([Hex-Hex+Na]⁺), consistent with a Hex-HexNol-HexN-Hex-Hex or a core 2 with a type 2 elongation and a terminal Gal (Table II). Hence, both neutral and sialylated oligosaccharides potentially expressing terminal Galα1,3Gal is produced by the C2-1-9 clone, although the sialidated (NeuAc) structure seem to be the most abundant one. In addition to this, several sialidated oligosaccharides without terminal Hex-Hex (Galα1-3Gal) can be seen, but at a lower relative abundance (FIG. 9 and Table 2).

TABLE 2
Sequences and tentative structures of PSGL-1/mIgG_{2b} derived O-glycans

| | MW | | Clone | Clone |
|---|---|---|---|---|
| Sequence | kDa | Tentative structure | 105L4-1 | C2-1-9 |
| Hex-HexNol-HexN | 779.5 | Galβ1-3[GlcNAcβ1-6]GalNAcol | | X |
| NeuAc-Hex-HexNolNeuAc- | 895.4 | NeuAcα2-3Galβ1-3GalNAcol Galβ1- | X | X |
| HexNol-Hex | | 3[NeuAcα2-6]GalNAcol | | |
| NeuGc-Hex-HexNol | 925.5 | NeuGcα2-3Galβ1-3GalNAcol | X | X |
| Hex-HexNol-HexN-Hex | 983.5 | Galβ1-3[Galβ1-4GlcNAcβ1- | | X |
| (Hex-Hex-HexN-HexNol) | | 6]GalNol | | |
| NeuAc-Hex-HexNol-HexN | 1140.5 | NeuAcα2-3Galβ1-3[GlcNAcβ1- | | X |
| | | 6]GalNol | | |
| Hex-HexNol-HexN-Hex- | 1187.6 | Galβ1-3[Galα1-3Galβ1-4GlcNAcβ1- | | X |
| Hex | | 6] GalNacol | | |
| NeuAc-Hex-HexNol-NeuAc | 1256.5 | NeuAcα2-3Galβ1-3[NeuAcα2-6] | X | X |
| | | GalNAcol | | |
| NeuGc-Hex-HexNol-NeuAc | 1286.5 | NeuGcα2-3Galβ1-3[NeuAcα2-6] | X | |
| NeuAc-Hex-HexNol-NeuGc | | GalNAcol | | |
| | | NeuAcα2-3Galβ1-3[NeuGcα2-6] | | |
| | | GalNAcol | | |
| NeuGc-Hex-HexNol-NeuGc | 1316.5 | NeuGcα2-3Galβ1-3[NeuGcα2-6] | X | |
| | | GalNAcol | | |
| Hex-HexNol-HexN-Hex- | 1344.6 | Galβ1-3[NeuAcα2-3Galβ1- | | X |
| NeuAcNeuAc-Hex-HexNol- | | 4GlcNAcβ1-6]GalNAcol | | |
| HexN-Hex | | NeuAcα2-3Galβ1-3[Galβ1- | | |
| | | 4GlcNAcβ1-6]GalNAcol | | |
| NeuAc-Hex-HexNol-HexN- | 1548.7 | NeuAcα2-3[Galα1-3Galβ1- | | X |
| Hex-Hex | | 4GlcNAcβ1-6]GalNAcol | | |
| NeuGc-Hex-HexNol-HexN- | 1578.7 | NeuGcα2-3[Galα1-3Galβ1- | | X |
| Hex-Hex | | 4GlcNAcβ1-6]GalNAcol | | |
| NeuAc-Hex-HexNol-HexN- | 1705.7 | NeuAcα2-3[NeuAcα2-3Galβ1- | | X |
| Hex-NeuAc | | 4GlcNAcβ1-6]GalNAcol | | |
| NeuGc-Hex-HexNol-HexN- | 1735.8 | NeuGcα2-3[NeuAcα2-3Galβ1- | | X |
| Hex-NeuAc NeuAc-Hex- | | 4GlcNAcβ1-6]GalNAcol NeuAcα2- | | |
| HexNol-HexN-Hex-NeuGc | | 3[NeuGcα2-3Galβ1-4GlcNAcβ1- | | |
| | | 6]GalNAcol | | |

### EXAMPLE 3

### Expression Vectors

Exemplary expression vectors useful in the production of the fusion polypeptides are as follows:

TABLE 3

Core 2 beta1-6 GlcNAc transferase Expression vector
(SEQ ID NO: 4; 4917 nucleotides)

| | |
|---|---|
| 1 | GGCGTAATCT GCTGCTTGCA AACAAAAAAA CCACCGCTAC CAGCGGTGGT |
| 51 | TTGTTTGCCG GATCAAGAGC TACCAACTCT TTTTCCGAAG GAACTGGCTT |
| 101 | CAGCAGAGCG CAGATACCAA ATACTGTCCT TCTAGTGTAG CCGTAGTTAG |
| 151 | GCCACCACTT CAAGAACTCT GTAGCACCGC CTACATACCT CGCTCTGCTA |
| 201 | ATCCTGTTAC CAGTGGCTGC TGCCAGTGGC GATAAGTCGT GTCTTACCGG |
| 251 | GTTGGACTCA AGACGATAGT TACCGGATAA GGCGCAGCGG TCGGGCTGAA |
| 301 | CGGGGGGTTC GTGCACACAG CCCAGCTTGG AGCGAACGAC CTACACCGAA |
| 351 | CTGAGATACC TACAGCGTGA GCTATGAGAA AGCGCCACGC TTCCCGAAGG |
| 401 | GAGAAAGGCG GACAGGTATC CGGTAAGCGG CAGGGTCGGA ACAGGAGAGC |
| 451 | GCACGAGGGA GCTTCCAGGG GGAAACGCCT GGTATCTTTA TAGTCCTGTC |
| 501 | GGGTTTCGCC ACCTCTGACT TGAGCGTCGA TTTTTGTGAT GCTCGTCAGG |
| 551 | GGGGCGGAGC CTATGGAAAA ACGCCAGCAA CGCCGAATTA CCGCGGTCTT |
| 601 | TCGGACTTTT GAAAGTGATG GTGGTGGGGG AAGGATTCGA ACCTTCGAAG |
| 651 | TCGATGACGG CAGATTTAGA GTCTGCTCCC TTTGGCCGCT CGGGAACCCC |
| 701 | ACCACGGGTA ATGCTTTTAC TGGCCTGCTC CCTTATCGGG AAGCGGGGCG |
| 751 | CATCATATCA AATGACGCGC CGCTGTAAAG TGTTACGTTG AGAAAGCTGC |
| 801 | TCCCTGCTTG TGTGTTGGAG GTCGCTGAGT AGTGCGCGAG TAAAATTTAA |
| 851 | GCTACAACAA GGCAAGGCTT GACCGACAAT TGCATGAAGA ATCTGCTTAG |
| 901 | GGTTAGGCGT TTTGCGCTGC TTCGGactag tGAGGCTCCG GTGCCCGTCA |
| 951 | GTGGGCAGAG CGCACATCGC CCACAGTCCC CGAGAAGTTG GGGGGAGGGG |
| 1001 | TCGGCAATTG AACCGGTGCC TAGAGAAGGT GGCGCGGGGT AAACTGGGAA |
| 1051 | AGTGATGTCG TGTACTGGCT CCGCCTTTTT CCCGAGGGTG GGGGAGAACC |
| 1101 | GTATATAAGT GCAGTAGTCG CCGTGAACGT TCTTTTTCGC AACGGGTTTG |
| 1151 | CCGCCAGAAC ACAGGTAAGT GCCGTGTGTG GTTCCCGCGG GCCTGGCCTC |
| 1201 | TTTACGGGTT ATGGCCCTTG CGTGCCTTGA ATTACTTCCA CGCCCCTGGC |
| 1251 | TGCAGTACGT GATTCTTGAT CCCGAGCTTC GGGTTGGAAG TGGGTGGGAG |
| 1301 | AGTTCGAGGC CTTGCGCTTA AGGAGCCCCT TCGCCTCGTG CTTGAGTTGA |
| 1351 | GGCCTGGCCT GGGCGCTGGG GCCGCCGCGT GCGAATCTGG TGGCACCTTC |
| 1401 | GCGCCTGTCT CGCTGCTTTC GATAAGTCTC TAGCCATTTA AAATTTTTGA |
| 1451 | TGACCTGCTG CGACGCTTTT TTTCTGGCAA GATAGTCTTG TAAATGCGGG |
| 1501 | CCAAGATCTG CACACTGGTA TTTCGGTTTT TGGGGCCGCG GGCGGCGACG |
| 1551 | GGGCCCGTGC GTCCCAGCGC ACATGTTCGG CGAGGCGGGG CCTGCGAGCG |
| 1601 | CGGCCACCGA GAATCGGACG GGGGTAGTCT CAAGCTGGCC GGCCTGCTCT |
| 1651 | GGTGCCTGGC CTCGCGCCGC CGTGTATCGC CCCGCCCTGG GCGGCAAGGC |
| 1701 | TGGCCCGGTC GGCACCAGTT GCGTGAGCGG AAAGATGGCC GCTTCCCGGC |
| 1751 | CCTGCTGCAG GGAGCTCAAA ATGGAGGACG CGGCGCTCGG GAGAGCGGGC |
| 1801 | GGGTGAGTCA CCCACACAAA GGAAAAGGGC CTTTCCGTCC TCAGCCGTCG |
| 1851 | CTTCATGTGA CTCCACGGAG TACCGGGCGC CGTCCAGGCA CCTCGATTAG |
| 1901 | TTCTCGAGCT TTTGGAGTAC GTCGTCTTTA GGTTGGGGGG AGGGGTTTTA |
| 1951 | TGCGATGGAG TTTCCCCACA CTGAGTGGGT GGAGACTGAA GTTAGGCCAG |
| 2001 | CTTGGCACTT GATGTAATTC TCCTTGGAAT TTGCCCTTTT TGAGTTTGGA |
| 2051 | TCTTGGTTCA TTCTCAAGCC TCAGACAGTG GTTCAAAGTT TTTTTCTTCC |
| 2101 | ATTTCAGGTG TCGTGAAAAG CTTCTAGAGA TCCCTCGACC TCGAGACCAT |
| 2151 | GCTGAGGACG TTGCTGCGAA GGAGACTTTT TTCTTATCCC ACCAAATACT |
| 2201 | ACTTTATGGT TCTTGTTTTA TCCCTAATCA CCTTCTCCGT TTTAAGGATT |
| 2251 | CATCAAAAGC CTGAATTTGT AAGTGTCAGA CACTTGGAGC TTGCTGGGGA |
| 2301 | GAATCCTAGT AGTGATATTA ATTGCACCAA AGTTTTACAG GGTGATGTAA |
| 2351 | ATGAAATCCA AAAGGTAAAG CTTGAGATCC TAACAGTGAA ATTTAAAAAG |
| 2401 | CGCCCTCGGT GGACACCTGA CGACTATATA AACATGACCA GTGACTGTTC |
| 2451 | TTCTTTCATC AAGAGACGCA AATATATTGT AGAACCCCTT AGTAAAGAAG |
| 2501 | AGGCGGAGTT TCCAATAGCA TATTCTATAG TGGTTCATCA CAAGATTGAA |
| 2551 | ATGCTTGACA GGCTGCTGAG GGCCATCTAT ATGCCTCAGA ATTTCTATTG |
| 2601 | CGTTCATGTG GACACAAAAT CCGAGGATTC CTATTTAGCT GCAGTGATGG |
| 2651 | GCATCGCTTC CTGTTTTAGT AATGTCTTTG TGGCCAGCCG ATTGGAGAGT |
| 2701 | GTGGTTTATG CATCGTGGAG CCGGGTTCAG GCTGACCTCA ACTGCATGAA |
| 2751 | GGATCTCTAT GCAATGAGTG CAAACTGGAA GTACTTGATA AATCTTTGTG |
| 2801 | GTATGGATTT TCCCATTAAA ACCAACCTAG AAATTGTCAG GAAGCTCAAG |
| 2851 | TTGTTAATGG GAGAAAACAA CCTGGAAACG GAGAGGATGC CATCCCATAA |
| 2901 | AGAAGAAAGG TGGAAGAAGC GGTATGAGGT CGTTAATGGA AAGCTGACAA |
| 2951 | ACACAGGGAC TGTCAAAATG CTTCCTCCAC TCGAAACACC TCTCTTTTCT |
| 3001 | GGCAGTGCCT ACTTCGTGGT CAGTAGGGAG TATGTGGGGT ATGTACTACA |
| 3051 | GAATGAAAAA ATCCAAAAGT TGATGGAGTG GGCACAAGAC ACATACAGCC |
| 3101 | CTGATGAGTA TCTCTGGGCC ACCATCCAAA GGATTCCTGA AGTCCCGGGC |
| 3151 | TCACTCCCTG CCAGCCATAA GTATGATCTA TCTGACATGC AAGCAGTTGC |
| 3201 | CAGGTTTGTC AAGTGGCAGT ACTTTGAGGG TGATGTTTCC AAGGGTGCTC |
| 3251 | CCTACCCGCC CTGCGATGGA GTCCATGTGC GCTCAGTGTG CATTTTCGGA |
| 3301 | GCTGGTGACT TGAACTGGAT GCTGCGCAAA CACCACTTGT TTGCCAATAA |
| 3351 | GTTTGACGTG GATGTTGACC TCTTTGCCAT CCAGTGTTTG GATGAGCATT |
| 3401 | TGAGACACAA AGCTTTGGAG ACATTAAAAC ACTGAGCGGC CGCCGCAGGT |
| 3451 | AAGCCAGCCC AGGCCTCGCC CTCCAGCTCA AGGCGGGACA GGTGCCCTAG |
| 3501 | AGTAGCCTGC ATCCAGGGAC AGGCCCCAGC CGGGTGCTGA CACGTCCACC |
| 3551 | TCCATCTCTT CCTCAGTTAA CTTGTTTATT GCAGCTTATA ATGGTTACAA |
| 3601 | ATAAAGCAAT AGCATCACAA ATTTCACAAA TAAAGCATTT TTTTCACTGC |
| 3651 | ATTCTAGTTG TGGTTTGTCC AAACTCATCA ATGTATCTTA TCATGTCTGG |
| 3701 | ATCCTCAGAA GAACTCGTCA AGAAGGCGAT AGAAGGCGAT GCGCTGCGAA |
| 3751 | TCGGGAGCGG CGATACCGTA AAGCACGAGG AAGCGGTCAG CCCATTCGCC |
| 3801 | GCCAAGCTCT TCAGCAATAT CACGGGTAGC CAACGCTATG TCCTGATAGC |
| 3851 | GGTCCGCCAC ACCCAGCCGG CCACAGTCGA TGAATCCAGA AAAGCGGCCA |
| 3901 | TTTTCCACCA TGATATTCGG CAAGCAGGCA TCGCCATGGG TCACGACGAG |
| 3951 | ATCCTCGCCG TCGGGCATGC GCGCCTTGAG CCTGGCGAAC AGTTCGGCTG |
| 4001 | GCGCGAGCCC CTGATGCTCT TCGTCCAGAT CATCCTGATC GACAAGACCG |
| 4051 | GCTTCCATCC GAGTACGTGC TCGCTCGATG CGATGTTTCG CTTGGTGGTC |
| 4101 | GAATGGGCAG GTAGCCGGAT CAAGCGTATG CAGCCGCCGC ATTGCATCAG |
| 4151 | CCATGATGGA TACTTTCTCG GCAGGAGCAA GGTGAGATGA CAGGAGATCC |
| 4201 | TGCCCCGGCA CTTCGCCCAA TAGCAGCCAG TCCCTTCCCG CTTCAGTGAC |
| 4251 | AACGTCGAGC ACAGCTGCGC AAGGAACGCC CGTCGTGGCC AGCCACGATA |
| 4301 | GCCGCGCTGC CTCGTCCTGC AGTTCATTCA GGGCACCGGA CAGGTCGGTC |
| 4351 | TTGACAAAAA GAACCGGGCG CCCCTGCGCT GACAGCCGGA ACACGGCGGC |
| 4401 | ATCAGAGCAG CCGATTGTCT GTTGTGCCCA GTCATAGCCG AATAGCCTCT |
| 4451 | CCACCCAAGC GGCCGGAGAA CCTGCGTGCA ATCCATCTTG TTCAATCATG |
| 4501 | GTCCTGCAGA GTCGCTCGGT GTTCGAGGCC ACACGCGTCA CCTTAATATG |
| 4551 | CGAAGTGGAC CTGGGACCGC GCCGCCCCGA CTGCATCTGC GTGTTCGAAT |
| 4601 | TCGCCAATGA CAAGACGCTG GGCGGGGTTT GTGTCATCAT AGAACTAAAG |
| 4651 | ACATGCAAAT ATATTTCTTC CGGGGACACC GCCAGCAAAC GCGAGCAACG |
| 4701 | GGCCACGGGG ATGAAGCAGC TGCGCCACTC CCTGAAGATC TCCCGCCCCT |
| 4751 | AACTCCGCCC ATCCCGCCCC TAACTCCGCC CAGTTCCGCC CATTCTCCGC |
| 4801 | CCCATGGCTG ACTAATTTTT TTTATTTATG CAGAGGCCGA GGCCGCGGCC |
| 4851 | TCTGAGCTAT TCCAGAAGTA GTGAGGAGGC TTTTTTGGAG GCCTAGGCTT |
| 4901 | TTGCAAAAAG CTAATTC |

TABLE 4

| | Corresponding | |
|---|---|---|
| | Nucleotide position in | |
| Nucleic Acid | SEQ ID NO: 1 | SEQ ID NO |
| pMB1 origin (pBR322 ori) | 1-593 | 5 |
| sac2) synthetic tyrosine | 594-925 | 6 |
| suppressor tRNA gene(supF | | |
| gene)remnant of ASV LTR | | |
| (spe) EF1alpha prom | 926-2139 | 7 |
| (xho) Core 2 beta1-6 GlcNAc | 2140-3435 | 8 |
| transferase 1 | | |
| (not) IgG1 hinge/CH2 intron | 3436-3565 | 9 |
| (hpa1) SV40 poly A | 3566-3698 | 10 |
| (bamh1) neomycin (rev) | 3699-4503 | 11 |
| (pst) HSV1 tk promoter −215 | 4504-4735 | 12 |
| to +19, with G to A mutation | | |
| at +7 | | |
| (bg12) SV40 origin (minus | 4736-4917 | 13 |
| enhancer) | | |

TABLE 5

Porcine α1,3 Galactosyltransferase Expression vector
(SEQ ID NO: 14; 4930 nucleotides)

| | |
|---|---|
| 1 | GGCGTAATCT GCTGCTTGCA AACAAAAAAA CCACCGCTAC CAGCGGTGGT |
| 51 | TTGTTTGCCG GATCAAGAGC TACCAACTCT TTTTCCGAAG GAACTGGCTT |
| 101 | CAGCAGAGCG CAGATACCAA ATACTGTCCT TCTAGTGTAG CCGTAGTTAG |
| 151 | GCCACCACTT CAAGAACTCT GTAGCACCGC CTACATACCT CGCTCTGCTA |
| 201 | ATCCTGTTAC CAGTGGCTGC TGCCAGTGGC GATAAGTCGT GTCTTACCGG |
| 251 | GTTGGACTCA AGACGATAGT TACCGGATAA GGCGCAGCGG TCGGGCTGAA |
| 301 | CGGGGGGTTC GTGCACACAG CCCAGCTTGG AGCGAACGAC CTACACCGAA |
| 351 | CTGAGATACC TACAGCGTGA GCTATGAGAA AGCGCCACGC TTCCCGAAGG |
| 401 | GAGAAAGGCG GACAGGTATC CGGTAAGCGG CAGGGTCGGA ACAGGAGAGC |
| 451 | GCACGAGGGA GCTTCCAGGG GGAAACGCCT GGTATCTTTA TAGTCCTGTC |
| 501 | GGGTTTCGCC ACCTCTGACT TGAGCGTCGA TTTTTGTGAT GCTCGTCAGG |
| 551 | GGGGCGGAGC CTATGGAAAA ACGCCAGCAA CGCCGAATTA CCGCGGTCTT |
| 601 | TCGGACTTTT GAAAGTGATG GTGGTGGGGG AAGGATTCGA ACCTTCGAAG |
| 651 | TCGATGACGG CAGATTTAGA GTCTGCTCCC TTTGGCCGCT CGGGAACCCC |
| 701 | ACCACGGGTA ATGCTTTTAC TGGCCTGCTC CCTTATCGGG AAGCGGGGCG |
| 751 | CATCATATCA AATGACGCGC CGCTGTAAAG TGTTACGTTG AGAAAGCTGC |
| 801 | TCCCTGCTTG TGTGTTGGAG GTCGCTGAGT AGTGCGCGAG TAAAATTTAA |
| 851 | GCTACAACAA GGCAAGGCTT GACCGACAAT TGCATGAAGA ATCTGCTTAG |
| 901 | GGTTAGGCGT TTTGCGCTGC TTCGGactag tGAGGCTCCG GTGCCCGTCA |
| 951 | GTGGGCAGAG CGCACATCGC CCACAGTCCC CGAGAAGTTG GGGGGAGGGG |
| 1001 | TCGGCAATTG AACCGGTGCC TAGAGAAGGT GGCGCGGGGT AAACTGGGAA |
| 1051 | AGTGATGTCG TGTACTGGCT CCGCCTTTTT CCCGAGGGTG GGGGAGAACC |
| 1101 | GTATATAAGT GCAGTAGTCG CCGTGAACGT TCTTTTTCGC AACGGGTTTG |
| 1151 | CCGCCAGAAC ACAGGTAAGT GCCGTGTGTG GTTCCCGCGG GCCTGGCCTC |
| 1201 | TTTACGGGTT ATGGCCCTTG CGTGCCTTGA ATTACTTCCA CGCCCCTGGC |
| 1251 | TGCAGTACGT GATTCTTGAT CCCGAGCTTC GGGTTGGAAG TGGGTGGGAG |
| 1301 | AGTTCGAGGC CTTGCGCTTA AGGAGCCCCT TCGCCTCGTG CTTGAGTTGA |
| 1351 | GGCCTGGCCT GGGCGCTGGG GCCGCCGCGT GCGAATCTGG TGGCACCTTC |
| 1401 | GCGCCTGTCT CGCTGCTTTC GATAAGTCTC TAGCCATTTA AAATTTTTGA |
| 1451 | TGACCTGCTG CGACGCTTTT TTTCTGGCAA GATAGTCTTG TAAATGCGGG |
| 1501 | CCAAGATCTG CACACTGGTA TTTCGGTTTT TGGGGCCGCG GGCGGCGACG |
| 1551 | GGGCCCGTGC GTCCCAGCGC ACATGTTCGG CGAGGCGGGG CCTGCGAGCG |
| 1601 | CGGCCACCGA GAATCGGACG GGGGTAGTCT CAAGCTGGCC GGCCTGCTCT |
| 1651 | GGTGCCTGGC CTCGCGCCGC CGTGTATCGC CCCGCCCTGG GCGGCAAGGC |
| 1701 | TGGCCCGGTC GGCACCAGTT GCGTGAGCGG AAAGATGGCC GCTTCCCGGC |
| 1751 | CCTGCTGCAG GGAGCTCAAA ATGGAGGACG CGGCGCTCGG GAGAGCGGGC |
| 1801 | GGGTGAGTCA CCCACACAAA GGAAAAGGGC CTTTCCGTCC TCAGCCGTCG |
| 1851 | CTTCATGTGA CTCCACGGAG TACCGGGCGC CGTCCAGGCA CCTCGATTAG |
| 1901 | TTCTCGAGCT TTTGGAGTAC GTCGTCTTTA GGTTGGGGGG AGGGGTTTTA |
| 1951 | TGCGATGGAG TTTCCCCACA CTGAGTGGGT GGAGACTGAA GTTAGGCCAG |
| 2001 | CTTGGCACTT GATGTAATTC TCCTTGGAAT TTGCCCTTTT TGAGTTTGGA |
| 2051 | TCTTGGTTCA TTCTCAAGCC TCAGACAGTG GTTCAAAGTT TTTTTCTTCC |
| 2101 | ATTTCAGGTG TCGTGAAaag cttaccATGA ATGTCAAAGG AAGAGTGGTT |
| 2151 | CTGTCAATGC TGCTTGTCTC AACTGTAATG GTTGTGTTTT GGGAATACAT |
| 2201 | CAACAGAAAC CCAGAAGTTG GCAGCAGTGC TCAGAGGGGC TGGTGGTTTC |
| 2251 | CGAGCTGGTT TAACAATGGG ACTCACAGTT ACCACGAAGA AGAAGACGCT |
| 2301 | ATAGGCAACG AAAAGGAACA AAGAAAAGAA GACAACAGAG GAGAGCTTCC |
| 2351 | GCTAGTGGAC TGGTTTAATC CTGAGAAACG CCCAGAGGTC GTGACCATAA |
| 2401 | CCAGATGGAA GGCTCCAGTG GTATGGGAAG GCACTTACAA CAGAGCCGTC |
| 2451 | TTAGATAATT ATTATGCCAA ACAGAAAATT ACCGTGGGCT TGACGGTTTT |
| 2501 | TGCTGTCGGA AGATACATTG AGCATTACTT GGAGGAGTTC TTAATATCTG |
| 2551 | CAAATACATA CTTCATGGTT GGCCACAAAG TCATCTTTTA CATCATGGTG |
| 2601 | GATGATATCT CCAGGATGCC TTTGATAGAG CTGGGTCCTC TGCGTTCCTT |
| 2651 | TAAAGTGTTT GAGATCAAGT CCGAGAAGAG GTGGCAAGAC ATCAGCATGA |
| 2701 | TGCGCATGAA GACCATCGGG GAGCACATCC TGGCCCACAT CCAGCACGAG |
| 2751 | GTGGACTTCC TCTTCTGCAT TGACGTGGAT CAGGTCTTCC AAAACAACTT |
| 2801 | TGGGGTGGAG ACCCTGGGCC AGTCGGTGGC TCAGCTACAG GCCTGGTGGT |
| 2851 | ACAAGGCACA TCCTGACGAG TTCACCTACG AGAGGCGGAA GGAGTCCGCA |
| 2901 | GCCTACATTC CGTTTGGCCA GGGGGATTTT TATTACCACG CAGCCATTTT |
| 2951 | TGGGGGAACA CCCACTCAGG TTCTAAACAT CACTCAGGAG TGCTTCAAGG |
| 3001 | GAATCCTCCA GGACAAGGAA AATGACATAG AAGCCGAGTG GCATGATGAA |
| 3051 | AGCCATCTAA ACAAGTATTT CCTTCTCAAC AAACCCACTA AAATCTTATC |
| 3101 | CCCAGAATAC TGCTGGGATT ATCATATAGG CATGTCTGTG GATATTAGGA |
| 3151 | TTGTCAAGAT AGCTTGGCAG AAAAAAGAGT ATAATTTGGT TAGAAATAAC |
| 3201 | ATCTGAgcgg ccgcCGCAGG TAAGCCAGCC CAGGCCTCGC CCTCCAGCTC |
| 3251 | AAGGCGGGAC AGGTGCCCTA GAGTAGCCTG CATCCAGGGA CAGGCCCCAG |
| 3301 | CCGGGTGCTG ACACGTCCAC CTCCATCTCT TCCTCAGTTA ACTTGTTTAT |
| 3351 | TGCAGCTTAT AATGGTTACA AATAAAGCAA TAGCATCACA AATTTCACAA |
| 3401 | ATAAAGCATT TTTTTCACTG CATTCTAGTT GTGGTTTGTC CAAACTCATC |
| 3451 | AATGTATCTT ATCATGTCTg gatccGCTAG CGCTTTATTC CTTTGCCCTC |
| 3501 | GGACGAGTGC TGGGGCGTCG GTTTCCACTA TCGGCGAGTA CTTCTACACA |
| 3551 | GCCATCGGTC CAGACGGCCG CGCTTCTGCG GGCGATTTGT GTACGCCCGA |
| 3601 | CAGTCCCGGC TCCGGATCGG ACGATTGCGT CGCATCGACC CTGCGCCCAA |
| 3651 | GCTGCATCAT CGAAATTGCC GTCAACCAAG CTCTGATAGA GTTGGTCAAG |
| 3701 | ACCAATGCGG AGCATATACG CCCGGAGCCG CGGCGATCCT GCAAGCTCCG |
| 3751 | GATGCCTCCG CTCGAAGTAG CGCGTCTGCT GCTCCATACA AGCCAACCAC |
| 3801 | GGCCTCCAGA AGAAGATGTT GGCGACCTCG TATTGGGAAT CCCCGAACAT |
| 3851 | CGCCTCGCTC CAGTCAATGA CCGCTGTTAT GCGGCCATTG TCCGTCAGGA |
| 3901 | CATTGTTGGA GCCGAAATCC GCGTGCACGA GGTGCCGGAC TTCGGGGCAG |
| 3951 | TCCTCGGCCC AAAGCATCAG CTCATCGAGA GCCTGCGCGA CGGACGCACT |
| 4001 | GACGGTGTCG TCCATCACAG TTTGCCAGTG ATACACATGG GGATCAGCAA |
| 4051 | TCGCGCATAT GAAATCACGC CATGTAGTGT ATTGACCGAT TCCTTGCGGT |
| 4101 | CCGAATGGGC CGAACCCGCT CGTCTGGCTA AGATCGGCCG CAGCGATCGC |
| 4151 | ATCCATCGCC TCCGCGACCG GCTGCAGAAC AGCGGGCAGT TCGGTTTCAG |
| 4201 | GCAGGTCTTG CAACGTGACA CCCTGTGCAC GGCGGGAGAT GCAATAGGTC |
| 4251 | AGGCTCTCGC TGAATTCCCC AATGTCAAGC ACTTCCGGAA TCGGGAGCGC |
| 4301 | GGCCGATGCA AAGTGCCGAT AAACATAACG ATCTTTGTAG AAACCATCGG |
| 4351 | CGCAGCTATT TACCCGCAGG ACATATCCAC GCCCTCCTAC ATCGAAGCTG |
| 4401 | AAAGCACGAG ATTCTTCGCC CTCCGAGAGC TGCATCAGGT CGGAGACGCT |
| 4451 | GTCGAACTTT TCGATCAGAA ACTTCTCGAC AGACGTCGCG GTGAGTTCAG |
| 4501 | GCTTTTTCAT GGTGGCCTGC AGAGTCGCTC GGTGTTCGAG GCCACACGCG |
| 4551 | TCACCTTAAT ATGCGAAGTG GACCTGGGAC CGCGCCGCCC CGACTGCATC |
| 4601 | TGCGTGTTCG AATTCGCCAA TGACAAGACG CTGGGCGGGG TTTGTGTCAT |
| 4651 | CATAGAACTA AAGACATGCA AATATATTTC TTCCGGGGAC ACCGCCAGCA |
| 4701 | AACGCGAGCA ACGGGCCACG GGGATGAAGC AGCTGCGCCA CTCCCTGAAG |
| 4751 | ATCTCCCGCC CCTAACTCCG CCCATCCCGC CCCTAACTCC GCCCAGTTCC |
| 4801 | GCCCATTCTC CGCCCCATGG CTGACTAATT TTTTTTATTT ATGCAGAGGC |
| 4851 | CGAGGCCGCG GCCTCTGAGC TATTCCAGAA GTAGTGAGGA GGCTTTTTTG |
| 4901 | GAGGCCTAGG CTTTTGCAAA AAGCTAATTC |

TABLE 6

| | Corresponding | |
|---|---|---|
| | Nucleotide position in | |
| Nucleic Acid | SEQ ID NO: 11 | SEQ ID NO |
| pMB1 origin (pBR322 ori) | 1-593 | 15 |
| sac2) synthetic tyrosine | 594-925 | 16 |
| suppressor tRNA gene(supF | | |
| gene)remnant of ASV LTR | | |
| (spe) EF1alpha prom | 926-2117 | 17 |
| (hind3) porcine | 2118-3206 | 18 |
| alpha1,3galactosyltransferase | | |
| (not) IgG1 hinge/CH2 intron | 3207-3336 | 19 |
| (hpa1) SV40 poly A | 3337-3469 | 20 |
| (bamh1) hygromycin b (rev) | 3470-4503 | 21 |
| (pst) HSV1 tk promoter −215 | 4517-4748 | 22 |
| to +19, with G to A mutation | | |
| at +7 | | |
| (bg12) SV40 origin (minus | 4749-4930 | 23 |
| enhancer) | | |

TABLE 7

Human PSGL-1 Expression vector (SEQ ID NO: 24; 5204
nucleotides)

| | |
|---|---|
| 1 | GGCGTAATCT GCTGCTTGCA AACAAAAAAA CCACCGCTAC CAGCGGTGGT |
| 51 | TTGTTTGCCG GATCAAGAGC TACCAACTCT TTTTCCGAAG GAACTGGCTT |
| 101 | CAGCAGAGCG CAGATACCAA ATACTGTCCT TCTAGTGTAG CCGTAGTTAG |
| 151 | GCCACCACTT CAAGAACTCT GTAGCACCGC CTACATACCT CGCTCTGCTA |
| 201 | ATCCTGTTAC CAGTGGCTGC TGCCAGTGGC GATAAGTCGT GTCTTACCGG |
| 251 | GTTGGACTCA AGACGATAGT TACCGGATAA GGCGCAGCGG TCGGGCTGAA |
| 301 | CGGGGGGTTC GTGCACACAG CCCAGCTTGG AGCGAACGAC CTACACCGAA |
| 351 | CTGAGATACC TACAGCGTGA GCTATGAGAA AGCGCCACGC TTCCCGAAGG |
| 401 | GAGAAAGGCG GACAGGTATC CGGTAAGCGG CAGGGTCGGA ACAGGAGAGC |
| 451 | GCACGAGGGA GCTTCCAGGG GGAAACGCCT GGTATCTTTA TAGTCCTGTC |
| 501 | GGGTTTCGCC ACCTCTGACT TGAGCGTCGA TTTTTGTGAT GCTCGTCAGG |
| 551 | GGGGCGGAGC CTATGGAAAA ACGCCAGCAA CGCCGAATTA CCGCGGTCTT |
| 601 | TCGGACTTTT GAAAGTGATG GTGGTGGGGG AAGGATTCGA ACCTTCGAAG |
| 651 | TCGATGACGG CAGATTTAGA GTCTGCTCCC TTTGGCCGCT CGGGAACCCC |
| 701 | ACCACGGGTA ATGCTTTTAC TGGCCTGCTC CCTTATCGGG AAGCGGGGCG |
| 751 | CATCATATCA AATGACGCGC CGCTGTAAAG TGTTACGTTG AGAAAGCTGC |
| 801 | TCCCTGCTTG TGTGTTGGAG GTCGCTGAGT AGTGCGCGAG TAAAATTTAA |
| 851 | GCTACAACAA GGCAAGGCTT GACCGACAAT TGCATGAAGA ATCTGCTTAG |
| 901 | GGTTAGGCGT TTTGCGCTGC TTCGGactag tGAGGCTCCG GTGCCCGTCA |
| 951 | GTGGGCAGAG CGCACATCGC CCACAGTCCC CGAGAAGTTG GGGGGAGGGG |
| 1001 | TCGGCAATTG AACCGGTGCC TAGAGAAGGT GGCGCGGGGT AAACTGGGAA |
| 1051 | AGTGATGTCG TGTACTGGCT CCGCCTTTTT CCCGAGGGTG GGGGAGAACC |
| 1101 | GTATATAAGT GCAGTAGTCG CCGTGAACGT TCTTTTTCGC AACGGGTTTG |
| 1151 | CCGCCAGAAC ACAGGTAAGT GCCGTGTGTG GTTCCCGCGG GCCTGGCCTC |
| 1201 | TTTACGGGTT ATGGCCCTTG CGTGCCTTGA ATTACTTCCA CGCCCCTGGC |
| 1251 | TGCAGTACGT GATTCTTGAT CCCGAGCTTC GGGTTGGAAG TGGGTGGGAG |
| 1301 | AGTTCGAGGC CTTGCGCTTA AGGAGCCCCT TCGCCTCGTG CTTGAGTTGA |
| 1351 | GGCCTGGCCT GGGCGCTGGG GCCGCCGCGT GCGAATCTGG TGGCACCTTC |
| 1401 | GCGCCTGTCT CGCTGCTTTC GATAAGTCTC TAGCCATTTA AAATTTTTGA |
| 1451 | TGACCTGCTG CGACGCTTTT TTTCTGGCAA GATAGTCTTG TAAATGCGGG |
| 1501 | CCAAGATCTG CACACTGGTA TTTCGGTTTT TGGGGCCGCG GGCGGCGACG |
| 1551 | GGGCCCGTGC GTCCCAGCGC ACATGTTCGG CGAGGCGGGG CCTGCGAGCG |
| 1601 | CGGCCACCGA GAATCGGACG GGGGTAGTCT CAAGCTGGCC GGCCTGCTCT |
| 1651 | GGTGCCTGGC CTCGCGCCGC CGTGTATCGC CCCGCCCTGG GCGGCAAGGC |
| 1701 | TGGCCCGGTC GGCACCAGTT GCGTGAGCGG AAAGATGGCC GCTTCCCGGC |
| 1751 | CCTGCTGCAG GGAGCTCAAA ATGGAGGACG CGGCGCTCGG GAGAGCGGGC |
| 1801 | GGGTGAGTCA CCCACACAAA GGAAAAGGGC CTTTCCGTCC TCAGCCGTCG |
| 1851 | CTTCATGTGA CTCCACGGAG TACCGGGCGC CGTCCAGGCA CCTCGATTAG |
| 1901 | TTCTCGAGCT TTTGGAGTAC GTCGTCTTTA GGTTGGGGGG AGGGGTTTTA |
| 1951 | TGCGATGGAG TTTCCCCACA CTGAGTGGGT GGAGACTGAA GTTAGGCCAG |
| 2001 | CTTGGCACTT GATGTAATTC TCCTTGGAAT TTGCCCTTTT TGAGTTTGGA |
| 2051 | TCTTGGTTCA TTCTCAAGCC TCAGACAGTG GTTCAAAGTT TTTTTCTTCC |
| 2101 | ATTTCAGGTG TCGTGAAaag cTTCTAGAGA TCCCTCGACC TCGAGATCCA |
| 2151 | TTGTGCTCTA AAGGAGATAC CCGGCCAGAC ACCCTCACCT GCGGTGCCCA |
| 2201 | GCTGCCCAGG CTGAGGCAAG AGAAGGCCAG AAACCATGCC CATGGGGTCT |
| 2251 | CTGCAACCGC TGGCCACCTT GTACCTGCTG GGGATGCTGG TCGCTTCCGT |
| 2301 | GCTAGCGCAG CTGTGGGACA CCTGGGCAGA TGAAGCCGAG AAAGCCTTGG |
| 2351 | GTCCCCTGCT TGCCCGGGAC CGGAGACAGG CCACCGAATA TGAGTACCTA |
| 2401 | GATTATGATT TCCTGCCAGA AACGGAGCCT CCAGAAATGC TGAGGAACAG |
| 2451 | CACTGACACC ACTCCTCTGA CTGGGCCTGG AACCCCTGAG TCTACCACTG |
| 2501 | TGGAGCCTGC TGCAAGGCGT TCTACTGGCC TGGATGCAGG AGGGGCAGTC |
| 2551 | ACAGAGCTGA CCACGGAGCT GGCCAACATG GGGAACCTGT CCACGGATTC |
| 2601 | AGCAGCTATG GAGATACAGA CCACTCAACC AGCAGCCACG GAGGCACAGA |
| 2651 | CCACTCCACT GGCAGCCACA GAGGCACAGA CAACTCGACT GACGGCCACG |
| 2701 | GAGGCACAGA CCACTCCACT GGCAGCCACA GAGGCACAGA CCACTCCACC |
| 2751 | AGCAGCCACG GAAGCACAGA CCACTCAACC CACAGGCCTG GAGGCACAGA |
| 2801 | CCACTGCACC AGCAGCCATG GAGGCACAGA CCACTGCACC AGCAGCCATG |
| 2851 | GAAGCACAGA CCACTCCACC AGCAGCCATG GAGGCACAGA CCACTCAAAC |
| 2901 | CACAGCCATG GAGGCACAGA CCACTGCACC AGAAGCCACG GAGGCACAGA |
| 2951 | CCACTCAACC CACAGCCACG GAGGCACAGA CCACTCCACT GGCAGCCATG |
| 3001 | GAGGCCCTGT CCACAGAACC CAGTGCCACA GAGGCCCTGT CCATGGAACC |
| 3051 | TACTACCAAA AGAGGTCTGT TCATACCCTT TTCTGTGTCC TCTGTTACTC |
| 3101 | ACAAGGGCAT TCCCATGGCA GCCAGCAATT TGTCCGTCAA CTACCCAGTG |
| 3151 | GGGGCCCCAG ACCACATCTC TGTGAAGCAG GATCCCGAGC CCAGCGGGCC |
| 3201 | CATTTCAACA ATCAACCCCT GTCCTCCATG CAAGGAGTGT CACAAATGCC |
| 3251 | CAGCTCCTAA CCTCGAGGGT GGACCATCCG TCTTCATCTT CCCTCCAAAT |
| 3301 | ATCAAGGATG TACTCATGAT CTCCCTGACA CCCAAGGTCA CGTGTGTGGT |
| 3351 | GGTGGATGTG AGCGAGGATG ACCCAGACGT CCAGATCAGC TGGTTTGTGA |
| 3401 | ACAACGTGGA AGTACACACA GCTCAGACAC AAACCCATAG AGAGAATTAC |
| 3451 | AACAGTACTG TCCGGGTGGT CAGCACCCTC CCCATCCAGC ACCAGGACTG |
| 3501 | GATGAGTGGC AAGGAGTTCA AATGCAAGGT CAACAACAAA GACCTCCCAT |
| 3551 | CACCCATCGA GAGAACCATC TCAAAAATTA AAGGGCTAGT CAGAGCTCCA |
| 3601 | CAAGTATACA TCTTGCCGCC ACCAGCAGAG CAGTTGTCCA GGAAAGATGT |
| 3651 | CAGTCTCACT TGCCTGGTCG TGGGCTTCAA CCCTGGAGAC ATCAGTGTGG |
| 3701 | AGTGGACCAG CAATGGGCAT ACAGAGGAGA ACTATAAGGA CACCGCACCA |
| 3751 | GTCCTGGACT CTGACGGTTC TTACTTCATA TATAGCAAGC TCAATATGAA |
| 3801 | AACAAGCAAG TGGGAGAAAA CAGATTCCTT CTCATGCAAC GTGAGACACG |
| 3851 | AGGGTCTGAA AAATTACTAC CTAAAGAAGA CCATCTCCCG GTCTCCGGGT |
| 3901 | AAATGAgcgg ccgcCGCAGG TAAGCCAGCC CAGGCCTCGC CCTCCAGCTC |
| 3951 | AAGGCGGGAC AGGTGCCCTA GAGTAGCCTG CATCCAGGGA CAGGCCCCAG |
| 4001 | CCGGGTGCTG ACACGTCCAC CTCCATCTCT TCCTCAGTTA ACTTGTTTAT |
| 4051 | TGCAGCTTAT AATGGTTACA AATAAAGCAA TAGCATCACA AATTTCACAA |
| 4101 | ATAAAGCATT TTTTTCACTG CATTCTAGTT GTGGTTTGTC CAAACTCATC |
| 4151 | AATGTATCTT ATCATGTCTG GATCCGCTAG CGCTTCAGGC ACCGGGCTTG |
| 4201 | CGGGTCATGC ACCAGGTCGC GCGGTCCTTC GGGCACTCGA CGTCGGCGGT |
| 4251 | GACGGTGAAG CCGAGCCGCT CGTAGAAGGG GAGGTTGCGG GGCGCGGAGG |
| 4301 | TCTCCAGGAA GGCGGGCACC CCGGCGCGCT CGGCCGCCTC CACTCCGGGG |
| 4351 | AGCACGACGG CGCTGCCCAG ACCCTTGCCC TGGTGGTCGG GCGAGACGCC |
| 4401 | GACGGTGGCC AGGAACCACG CGGGCTCCTT GGGCCGGTGC GGCGCCAGGA |
| 4451 | GGCCTTCCAT CTGTTGCTGC GCGGCCAGCC GGGAACCGCT CAACTCGGCC |
| 4501 | ATGCGCGGGC CGATCTCGGC GAACACCGCC CCCGCTTCGA CGCTCTCCGG |
| 4551 | CGTGGTCCAG ACCGCCACCG CGGCGCCGTC GTCCGCGACC CACACCTTGC |
| 4601 | CGATGTCGAG CCCGACGCGC GTGAGGAAGA GTTCTTGCAG CTCGGTGACC |
| 4651 | CGCTCGATGT GGCGGTCCGG GTCGACGGTG TGGCGCGTGG CGGGGTAGTC |
| 4701 | GGCGAACGCG GCGGCGAGGG TGCGTACGGC CCGGGGGACG TCGTCGCGGG |
| 4751 | TGGCGAGGCG CACCGTGGGC TTGTACTCGG TCATGGTGGC CTGCAGAGTC |
| 4801 | GCTCGGTGTT CGAGGCCACA CGCGTCACCT TAATATGCGA AGTGGACCTG |
| 4851 | GGACCGCGCC GCCCCGACTG CATCTGCGTG TTCGAATTCG CCAATGACAA |
| 4901 | GACGCTGGGC GGGGTTTGTG TCATCATAGA ACTAAAGACA TGCAAATATA |
| 4951 | TTTCTTCCGG GGACACCGCC AGCAAACGCG AGCAACGGGC CACGGGGATG |
| 5001 | AAGCAGCTGC GCCACTCCCT GAAGATCTCC CGCCCCTAAC TCCGCCCATC |
| 5051 | CCGCCCCTAA CTCCGCCCAG TTCCGCCCAT TCTCCGCCCC ATGGCTGACT |
| 5101 | AATTTTTTTT ATTTATGCAG AGGCCGAGGC CGCGGCCTCT GAGCTATTCC |
| 5151 | AGAAGTAGTG AGGAGGCTTT TTTGGAGGCC TAGGCTTTTG CAAAAAGCTA |
| 5201 | ATTC |

TABLE 8

| | Corresponding | |
|---|---|---|
| | Nucleotide position in | |
| Nucleic Acid | SEQ ID NO: 21 | SEQ ID NO |
| pMB1 origin (pBR322 ori) | 1-593 | 25 |
| (sac2) synthetic tyrosine | 594-925 | 26 |
| suppressor tRNA gene(supF | | |
| gene)remnant of ASV LTR | | |
| (spe) EF1alpha prom | 926-2117 | 27 |
| (hind3) human PSGL-1/mouse | 2118-3906 | 28 |
| IgG2b | | |
| (not) IgG1 hinge/CH2 intron | 3907-4036 | 29 |
| (hpa1) SV40 poly A | 4037-4169 | 30 |
| (bamh1) puromycin | 4170-4790 | 31 |
| acetyltransferase | | |
| (pst) HSV1 tk promoter −215 | 4791-5022 | 32 |
| to +19, with G to A mutation | | |
| at +7 | | |
| (bg12) SV40 origin (minus | 5023-5204 | 33 |
| enhancer) | | |

### EXAMPLE 4

### Inhibiting Bacterial Toxin A Infection In Vitro

Toxin A and endothelial cells which express the carbohydrate receptor for the toxin are used to assess the inhibitory capacity of the above described fusion proteins with regards to preventing toxin-cell surface binding.

### EXAMPLE 5

### Routes of Administration

Recombinant PSGL-1/mIgG₂b carrying multiple Galα1,3Gal eptiopes (i.e., the αGal fusion protein) is administered systemically and/or rectally (e.g., rectal enema) to prevent/inhibit the effect of infection by Toxin A producing bacteria.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

### References

Wong C H. Enzymatic and Chemo-Enzymatic Synthesis Of Carbohydrates. Pure Appl. Chem. 1995; 67 (10): 1609-1616. 30
Seed B. An LFA-3 cDNA encodes a phospholipid-linked membrane protein homologous to its receptor CD2. Nature 1987; 329 (6142): 840-2.
Gustafsson K, Strahan K, Preece A. Alpha 1,3galactosyltransferase: a target for in vivo genetic manipulation in xenotransplantation. Immunol. Rev. 1994; 141: 59-70.
Sako D, Chang X J, Barone K M, et al. Expression cloning of a functional glycoprotein ligand for P-selectin. Cell 1993; 75 (6): 1179-86.
Laemmli U K. Cleavage of structural proteins during the assembly of the head of bacteriophage T4. Nature 1970; 227 (259): 680-5.
Towbin H, Staehelin T, Gordon J. Electrophoretic transfer of proteins from polyacrylamide gels to nitrocellulose sheets: procedure and some applications. Proc. Natl. Acad. Sci. USA 1979; 76 (9): 4350-4
Carraway KL, Hull SR. Cell surface mucin-type glycoproteins and mucin-like domains. Glycobiology 1991; 1 (2): 131-8.
Shimizu Y, Shaw S. Mucins in the mainstream. Nature 1993; 366: 630-631.
Galili U, Macher B A, Buehler J, Shohet S B. Human natural anti-a-galactosyl IgG. II. The specific recognition of a(I-3)-linked galactose residues. J. Exp. Med. 1985; 162: 573-582.
Rosen S D, Bertozzi C R. Leukocyte adhesion: Two selectins converge on sulphate. Curr. Biol. 1996; 6 (3): 261-264.
Asa D, Raycroft L, Ma L, et al. The P-Selectin Glycoprotein Ligand Functions As a Common Human Leukocyte Ligand For P- and E-Selectins. J. Biol. Chem. 1995; 270 (19): 11662-11670.
Gauldi J, Richards C, Lamontagne L. Fe receptors for IgA and other immunoglobulins on resident and activated alveolar macrophages. Mol. Immunol. 1983; 20: 1029-1037.
Monteiro RC, Kubagawa H, Cooper M. Cellular distribution, regula-tion, and biochemical nature of an Fca receptor in humans. J. Exp. Med. 1990; 17 1: 597-613.
Auchincloss, H., Jr., and Sachs, D. H. (1998). Xenogeneic transplantation. Annu Rev Immunol, 16, 433.
Bhatia, P. K., and Mukhopadhyay, A. (1998). Protein glycosyation: implications for in vivo functions and therapeutic applications. Adv Biochem Eng Biotechnol, 64, 155.
Bierhuizen, M. F., and Fukuda, M. (1992). Expression cloning of a cDNA encoding UDP-GlcNAc:Gal beta 1-3-GalNAc-R (GlcNAc to GalNAc) beta 1-6GlcNAc transferase by gene transfer into CHO cells expressing polyoma large tumor antigen. Proc Natl Acad Sci US A, 89, 9326.
Bouhours, D., Pourcel, C., and Bouhours, J. E. (1996). Simultaneous expression by porcine aorta endothelial cells of glycosphingolipids bearing the major epitope for human xenoreactive antibodies (Gal alpha 1-3 Gal), blood group H determinant and N-glycolylneuraminic acid. Glycoconj J. 13, 947.
Boussif, O., Lezoualc'h, F., Zanta, M. A., Mergny, M. D., Scherman, D., Demeneix, B., and Behr, J. P. (1995). A versatile vector for gene and oligonucleotide transfer into cells in culture and in vivo: polyethylenimine. Proc Natl Acad Sci USA, 92, 7297.
Cairns, T., Lee, J., Goldberg, L. C., Hakim, N., Cook, T., Rydberg, L., Samuelsson, B., and Taube, D. (1996). Thomsen-Friedenreich and PK antigens in pig-to-human xenotransplantation. Transplant Proc, 28, 795.
Cairns, T. D., Taube, D. H., Stevens, N., Binns, R., and Welsh, K. I. (1991). Xenografts—future prospects for clinical transplantation. Immunol Lett, 29, 167.
Carlstedt, I., Herrmann, A., Karlsson, H., Sheehan, J., Fransson, L. A., and Hansson, G. C. (1993). Characterization of two different glycosylated domains from the insoluble mucin complex of rat small intestine. J Biol Chem, 268, 18771.
Ciucanu, I., and Kerek, F. (1984). A simple and rapid method for the permethylation of carbohydrates. Carbohydr Res, 131, 209.
Corzana, F., Bettler, E., Herve du Penhoat, C., Tyrtysh, T. V., Bovin, N. V., and Imberty, A. (2002). Solution structure of two xenoantigens: alpha Gal-LacNAc and alpha Gal-Lewis X. Glycobiology, 12, 241.
Dennis, J. W. (1993). Core 2 GlcNAc-transferase and polylactosamine expression in O-glycans. Glycobiology, 3, 91.
Galili, U. (1993). Interaction of the natural anti-Gal antibody with alpha-galactosyl epitopes: a major obstacle for xenotransplantation in humans. Immunol Today, 14, 480.
Galili, U., Macher, B. A., Buehler, J., and Shohet, S. B. (1985). Human natural anti-alpha-galactosyl IgG. II. The specific recognition of alpha (1-3)-linked galactose residues. J Exp Med, 162, 573.
Gervais, A., Hammel, Y. A., Pelloux, S., Lepage, P., Baer, G., Carte, N., Sorokine, O., Strub, J. M., Koerner, R., Leize, E., and Van Dorsselaer, A. (2003). Glycosylation of human recombinant gonadotrophins: characterization and batch-to-batch consistency. Glycobiology, 13, 179.
Gustafsson, K., Strahan, K., and Preece, A. (1994). Alpha 1,3galactosyltransferase: a target for in vivo genetic manipulation in xenotransplantation. Immunol Rev, 141, 59.
Hansson, G. C., and Karlsson, H. (1993). Gas chromatography and gas chromatography-mass spectrometry of glycoprotein oligosaccharides. Methods Mol Biol, 14, 47.
He, Z., She, R., Sumitran-Holgersson, S., Blomberg, P., Islam, K. B., and Holgersson, J. (2001). The in vitro activity and specificity of human endothelial cell-specific promoters in porcine cells. Xenotransplantation, 8, 202.
Hokke, C. H., Bergwerff, A. A., Van Dedem, G. W., Kamerling, J. P., and Vliegenthart, J.

F. (1995). Structural analysis of the sialylated N- and O-linked carbohydrate chains of recombinant human erythropoietin expressed in Chinese hamster ovary cells. Sialylation patterns and branch location of dimeric N-acetyllactosamine units. Eur J Biochem, 228, 981.
Imberty, A., Mikros, E., Koca, J., Mollicone, R., Oriol, R., and Perez, S. (1995). Computer simulation of histo-blood group oligosaccharides: energy maps of all constituting disaccharides and potential energy surfaces of 14 ABH and Lewis carbohydrate antigens. Glycoconj J. 12, 331.
Imberty, A., Mollicone, R., Mikros, E., Carrupt, P. A., Perez, S., and Oriol, R. (1996).

How do antibodies and lectins recognize histo-blood group antigens? A 3D-QSAR study by comparative molecular field analysis (CoMFA). Bioorg Med Chem, 4, 1979.
Imberty, A., Perez, S., Hricovini, M., Shah, R. N., and Carver, J. P. (1993). Flexibility in a tetrasaccharide fragment from the high mannose type of N-linked oligosaccharides. Int J Biol Macromol, 15, 17.
Itoh, S., Kawasaki, N., Ohta, M., and Hayakawa, T. (2002). Structural analysis of a glycoprotein by liquid chromatography-mass spectrometry and liquid chromatography with tandem mass spectrometry. Application to recombinant human thrombomodulin. J Chromatogr A, 978, 141.
Joziasse, D. H., Shaper, J. H., Jabs, E. W., and Shaper, N. L. (1991). Characterization of an alpha 1-3-galactosyltransferase homologue on human chromosome 12 that is organized as a processed pseudogene. J Biol Chem, 266, 6991.
Kitov, P. I., Sadowska, J. M., Mulvey, G., Armstrong, G. D., Ling, H., Pannu, N. S., Read, R. J., and Bundle, D. R. (2000). Shiga-like toxins are neutralized by tailored multivalent carbohydrate ligands. Nature, 403, 669.
Kumar, R., Camphausen, R. T., Sullivan, F. X., and Cumming, D. A. (1996). Core2 beta-1,6-N-acetylglucosaminyltransferase enzyme activity is critical for P-selectin glycoprotein ligand-1 binding to P-selectin. Blood, 88, 3872.
Laemmli, U. K. (1970). Cleavage of structural proteins during the assembly of the head of bacteriophage T4. Nature, 227, 680.
Lee, R. T., and Lee, Y. C. (2000). Affinity enhancement by multivalent lectin-carbohydrate interaction. Glycoconj J. 17, 543.
Lindhorst, T. K., Kieburg, C., and Krallmann-Wenzel, U. (1998). Inhibition of the type 1 fimbriae-mediated adhesion of Escherichia coli to erythrocytes by multiantennary alpha-ERROR clusters: the effect of multivalency. Glycoconj J, 15, 605.
Liu, J., Qian, Y., and Holgersson, J. (1997). Removal of xenoreactive human anti-pig antibodies by absorption on recombinant mucin-containing glycoproteins carrying the Gal alphal,3Gal epitope. Transplantation, 63, 1673.
Liu, J., Weintraub, A., and Holgersson, J. (2003). Multivalent Galalphal,3Gal-substitution makes recombinant mucin-immunoglobulins efficient absorbers of anti-pig antibodies. Xenotransplantation, 10, 149.
Lofling, J. C., Hauzenberger, E., and Holgersson, J. (2002). Absorption of anti-blood group A antibodies on P-selectin glycoprotein ligand-1/immunoglobulin chimeras carrying blood group A determinants: core saccharide chain specificity of the Se and H gene encoded alpha1,2 fucosyltransferases in different host cells. Glycobiology, 12, 173.
Maaheimo, H., Renkonen, R., Turunen, J. P., Penttila, L., and Renkonen, O. (1995).

Synthesis of a divalent sialyl Lewis x O-glycan, a potent inhibitor of lymphocyte-endothelium adhesion. Evidence that multivalency enhances the saccharide binding to L-selectin. Eur J Biochem, 234, 616.
Malykh, Y. N., King, T. P., Logan, E., Kelly, D., Schauer, R., and Shaw, L. (2003). Regulation of N-glycolylneuraminic acid biosynthesis in developing pig small intestine. Biochem J. 370, 601.
Malykh, Y. N., Krisch, B., Shaw, L., Warner, T. G., Sinicropi, D., Smith, R., Chang, J., and Schauer, R. (2001). Distribution and localization of CMP-N-acetylneuraminic acid hydroxylase and N-glycolylneuraminic acid-containing glycoconjugates in porcine lymph node and peripheral blood lymphocytes. Eur J Cell Biol, 80, 48.
Mammen, M., Choi, S., and Whitesides, G. (1998). Polyvalent interactions in biological systems:implications for design and use of multivalent ligands and inhibitors. Angew Chem Int Ed, 37, 2754.
McEver, R. P., Moore, K. L., and Cummings, R. D. (1995). Leukocyte trafficking mediated by selectin-carbohydrate interactions. J Biol Chem, 270, 11025.
Mitoma, J., Petryniak, B., Hiraoka, N., Yeh, J. C., Lowe, J. B., and Fukuda, M. (2003). Extended core 1 and core 2 branched O-glycans differentially modulate sialyl Lewis X-type L-selectin ligand activity. J Biol Chem, 278, 9953.
Miyata, Y., and Platt, J. L. (2003). Xeno-still stuck without alphaGal. Nat Biotechnol, 21, 359.
Moore, K. L., Eaton, S. F., Lyons, D. E., Lichenstein, H. S., Cummings, R. D., and McEver, R. P. (1994). The P-selectin glycoprotein ligand from human neutrophils displays sialylated, fucosylated, O-linked poly-N-acetyllactosamine. J Biol Chem, 269,23318.
Neethling, F. A., Koren, E., Ye, Y., Richards, S. V., Kujundzic, M., Oriol, R., and Cooper, D. K. (1994). Protection of pig kidney (PK15) cells from the cytotoxic effect of anti-pig antibodies by alpha-galactosyl oligosaccharides. Transplantation, 57, 959.
Oriol, R., Ye, Y., Koren, E., and Cooper, D. K. (1993). Carbohydrate antigens of pig tissues reacting with human natural antibodies as potential targets for hyperacute vascular rejection in pig-to-man organ xenotransplantation. Transplantation, 56, 1433.
Pascher, A., Poehlein, C., Stangl, M., Thiery, J., Mueller-Derlich, J., and Hammer, C. (1997). Immunoapheresis, an advanced technique for depleting human anti-porcine antibodies, delays hyperacute rejection of xenogeneic perfused pig livers. Transplant Proc, 29, 962.
Phelps, C. J., Koike, C., Vaught, T. D., Boone, J., Wells, K. D., Chen, S. H., Ball, S., Specht, S. M., Polejaeva, I. A., Monahan, J. A., Jobst, P. M., Sharma, S. B., Lamborn, A. E., Garst, A. S., Moore, M., Demetris, A. J., Rudert, W. A., Bottino, R., Bertera, S., Trucco, M., Starzl, T. E., Dai, Y., and Ayares, D. L. (2003). Production of alpha 1,3-galactosyltransferase-deficient pigs. Science, 299, 411.
Renkonen, O., Toppila, S., Penttila, L., Salminen, H., Helin, J., Maaheimo, H., Costello, C. E., Turunen, J. P., and Renkonen, R. (1997). Synthesis of a new nanomolar saccharide inhibitor of lymphocyte adhesion: different polylactosamine backbones present multiple sialyl Lewis x determinants to L-selectin in high-affinity mode. Glycobiology, 7, 453.
Rydberg, L., Holgersson, J., Samuelsson, B. E., and Breimer, M. E. (1999). alpha-Gal epitopes in animal tissue glycoproteins and glycolipids. Subcell Biochem, 32, 107.
Sako, D., Chang, X. J., Barone, K. M., Vachino, G., White, H. M., Shaw, G., Veldman, G. M., Bean, K. M., Ahern, T. J., Furie, B., and et al. (1993). Expression cloning of a functional glycoprotein ligand for P-selectin. Cell, 75, 1179.
Sako, D., Comess, K. M., Barone, K. M., Camphausen, R. T., Cumming, D. A., and Shaw, G. D. (1995). A sulfated peptide segment at the amino terminus of PSGL-1 is critical for P-selectin binding. Cell, 83, 323.
Schachter, H. (2000). The joys of HexNAc. The synthesis and function of N- and O-glycan branches. Glycoconj J. 17, 465.
Sharma, A., Naziruddin, B., Cui, C., Martin, M. J., Xu, H., Wan, H., Lei, Y., Harrison, C., Yin, J., Okabe, J., Mathews, C., Stark, A., Adams, C. S., Houtz, J., Wiseman, B. S., Byrne, G. W., and Logan, J. S. (2003). Pig cells that lack the gene for alphal-3 galactosyltransferase express low levels of the gal antigen. Transplantation, 75, 430.
Sheeley, D. M., Merrill, B. M., and Taylor, L. C. (1997). Characterization of monoclonal antibody glycosylation: comparison of expression systems and identification of terminal alpha-linked galactose. Anal Biochem, 247, 102.
Silverman, H. S., Sutton-Smith, M., McDermott, K., Heal, P., Leir, S. H., Morris, H. R., Hollingsworth, M. A., Dell, A., and Harris, A. (2003). The contribution of tandem repeat number to the 0-glycosylation of mucins. Glycobiology, 13, 265.
Skrincosky, D., Kain, R., El-Battari, A., Exner, M., Kerjaschki, D., and Fukuda, M. (1997). Altered Golgi localization of core 2 beta-1,6-N-acetylglucosaminyltransferase leads to decreased synthesis of branched O-glycans. J Biol Chem, 272, 22695.
Ten Hagen, K. G., Fritz, T. A., and Tabak, L. A. (2003). All in the family: the UDP-GalNAc:polypeptide N-acetylgalactosaminyltransferases. Glycobiology, 13, 1.
Totani, K., Kubota, T., Kuroda, T., Murata, T., Hidari, K. I., Suzuki, T., Suzuki, Y., Kobayashi, K., Ashida, H., Yamamoto, K., and Usui, T. (2003). Chemoenzymatic synthesis and application of glycopolymers containing multivalent sialyloligosaccharides with a poly(L-glutamic acid) backbone for inhibition of infection by influenza viruses. Glycobiology, 13, 315.
Towbin, H., Staehelin, T., and Gordon, J. (1979). Electrophoretic transfer of proteins from polyacrylamide gels to nitrocellulose sheets: procedure and some applications. Proc Natl Acad Sci USA, 76, 4350.
Van den Nieuwenhof, I. M., Koistinen, H., Easton, R. L., Koistinen, R., Kamarainen, M., Morris, H. R., Van Die, I., Seppala, M., Dell, A., and Van den Eijnden, D. H. (2000). Recombinant glycodelin carrying the same type of glycan structures as contraceptive glycodelin-A can be produced in human kidney 293 cells but not in chinese hamster ovary cells. Eur J Biochem, 267, 4753.
Varki, A. (2001). N-glycolylneuraminic acid deficiency in humans. Biochimie, 83, 615. Yan, S. B., Chao, Y. B., and van Halbeek, H. (1993). Novel Asn-linked oligosaccharides terminating in GalNAc beta (1→4)[Fuc alpha (1→3)]GlcNAc beta (1→.) are present in recombinant human protein C expressed in human kidney 293 cells. Glycobiology, 3, 597.
Yeh, J. C., Ong, E., and Fukuda, M. (1999). Molecular cloning and expression of a novel beta-1, 6-N-acetylglucosaminyltransferase that forms core 2, core 4, and I branches. J Biol Chem, 274, 3215.
Zhu, A., and Hurst, R. (2002). Anti-N-glycolylneuraminic acid antibodies identified in healthy human serum. Xenotransplantation, 9, 376.

What is claimed is:

1. A method for preventing or alleviating a symptom of bacterial toxin infection in a subject in need thereof, the method comprising administering to the subject fusion polypeptide comprising a first polypeptide linked to a second polypeptide, wherein the first polypeptide:
(a) is a mucin polypeptide; and
(b) is glycosylated by an α1,3 galactosyltranserase and a β1,6, N-acetylglucosaminyltransferase; and
the second polypeptide comprises at least a region of an immunoglobulin polypeptide.

2. A method for preventing or alleviating a symptom of bacterial toxin infection in a subject in need thereof, the method comprising administering to the subject a fusion polypeptide comprising a first polypeptide linked to a second polypeptide, wherein
a) the fusion polypeptide comprises a glycan repertoire including one or more sequences, or a fragment thereof, selected from the group consisting of:
i) Hex-HexNol-HexN-Hex-Hex
ii) NeuAc-Hex-HexNol-HexN-Hex-Hex and
iii) NeuGc-Hex-HexNol-HexN-Hex-Hex; and
b) the second polypeptide comprises at least a region of an immunoglobulin polypeptide.

3. The method of claim 1 or 2, wherein the bacterial toxin is produced by Clostridium difficile.

4. The method of claim 1 or 2, wherein the bacterial toxin is C. difficile Toxin A.

5. The method of claim 1 or 2, wherein the fusion polypeptide is administered systemically.

6. The method of claim 1 or 2, wherein the fusion polypeptide is administered rectally.


SEQUENCE LISTING (34 sequences)

SEQ ID NO: 1 - protein, 10 aa, artificial sequence
  PSGL-1 consensus sequnece
XQTTXXXXXE

SEQ ID NO: 2 - DNA, 30 bp, artificial sequence
  synthetic primer
cgcgggctcgagatgaagatattcaaatgt

SEQ ID NO: 3 - DNA, 34 bp, artificial sequence
  synthetic primer
cgcggggcggccgctcatgatgtggtagtgagat

SEQ ID NO: 4 - DNA, 4917 bp, artificial sequence
  Core 2 beta 1-6 GlcNAc transferase expression vector
ggcgtaatctgctgcttgcaaacaaaaaaaccaccgctaccagcggtggtttgtttgccggatcaagagctaccaactctttttccgaaggaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgccgaattaccgcggtctttcggacttttgaaagtgatggtggtgggggaaggattcgaaccttcgaagtcgatgacggcagatttagagtctgctccctttggccgctcgggaaccccaccacgggtaatgcttttactggcctgctcccttatcgggaagcggggcgcatcatatcaaatgacgcgccgctgtaaagtgttacgttgagaaagctgctccctgcttgtgtgttggaggtcgctgagtagtgcgcgagtaaaatttaagctacaacaaggcaaggcttgaccgacaattgcatgaagaatctgcttagggttaggcgttttgcgctgcttcggactagtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaagcttctagagatccctcgacctcgagaccatgctgaggacgttgctgcgaaggagacttttttcttatcccaccaaatactactttatggttcttgttttatccctaatcaccttctccgttttaaggattcatcaaaagcctgaatttgtaagtgtcagacacttggagcttgctggggagaatcctagtagtgatattaattgcaccaaagttttacagggtgatgtaaatgaaatccaaaaggtaaagcttgagatcctaacagtgaaatttaaaaagcgccctcggtggacacctgacgactatataaacatgaccagtgactgttcttctttcatcaagagacgcaaatatattgtagaaccccttagtaaagaagaggcggagtttccaatagcatattctatagtggttcatcacaagattgaaatgcttgacaggctgctgagggccatctatatgcctcagaatttctattgcgttcatgtggacacaaaatccgaggattcctatttagctgcagtgatgggcatcgcttcctgttttagtaatgtctttgtggccagccgattggagagtgtggtttatgcatcgtggagccgggttcaggctgacctcaactgcatgaaggatctctatgcaatgagtgcaaactggaagtacttgataaatctttgtggtatggattttcccattaaaaccaacctagaaattgtcaggaagctcaagttgttaatgggagaaaacaacctggaaacggagaggatgccatcccataaagaagaaaggtggaagaagcggtatgaggtcgttaatggaaagctgacaaacacagggactgtcaaaatgcttcctccactcgaaacacctctcttttctggcagtgcctacttcgtggtcagtagggagtatgtggggtatgtactacagaatgaaaaaatccaaaagttgatggagtgggcacaagacacatacagccctgatgagtatctctgggccaccatccaaaggattcctgaagtcccgggctcactccctgccagccataagtatgatctatctgacatgcaagcagttgccaggtttgtcaagtggcagtactttgagggtgatgtttccaagggtgctccctacccgccctgcgatggagtccatgtgcgctcagtgtgcattttcggagctggtgacttgaactggatgctgcgcaaacaccacttgtttgccaataagtttgacgtggatgttgacctctttgccatccagtgtttggatgagcatttgagacacaaagctttggagacattaaaacactgagcggccgccgcaggtaagccagcccaggcctcgccctccagctcaaggcgggacaggtgccctagagtagcctgcatccagggacaggccccagccgggtgctgacacgtccacctccatctcttcctcagttaacttgtttattgcagcttataatggttacaaataaagcaatagcatcacaaatttcacaaataaagcatttttttcactgcattctagttgtggtttgtccaaactcatcaatgtatcttatcatgtctggatcctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatggtcctgcagagtcgctcggtgttcgaggccacacgcgtcaccttaatatgcgaagtggacctgggaccgcgccgccccgactgcatctgcgtgttcgaattcgccaatgacaagacgctgggcggggtttgtgtcatcatagaactaaagacatgcaaatatatttcttccggggacaccgccagcaaacgcgagcaacgggccacggggatgaagcagctgcgccactccctgaagatctcccgcccctaactccgcccatcccgcccctaactccgcccagttccgcccattctccgccccatggctgactaattttttttatttatgcagaggccgaggccgcggcctctgagctattccagaagtagtgaggaggcttttttggaggcctaggcttttgcaaaaagctaattc

SEQ ID NO: 5 - DNA, 593 bp, artificial sequence
  pMB1 origin (pBR322 ori)
ggcgtaatctgctgcttgcaaacaaaaaaaccaccgctaccagcggtggtttgtttgccggatcaagagctaccaactctttttccgaaggaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgccgaattaccg

SEQ ID NO: 6 - DNA, 332 bp, artificial sequence
  sac2 synthetic tyrosine suppressor tRNA gene (supF gene) remnant of ASV LTR
cggtctttcggacttttgaaagtgatggtggtgggggaaggattcgaaccttcgaagtcgatgacggcagatttagagtctgctccctttggccgctcgggaaccccaccacgggtaatgcttttactggcctgctcccttatcgggaagcggggcgcatcatatcaaatgacgcgccgctgtaaagtgttacgttgagaaagctgctccctgcttgtgtgttggaggtcgctgagtagtgcgcgagtaaaatttaagctacaacaaggcaaggcttgaccgacaattgcatgaagaatctgcttagggttaggcgttttgcgctgcttcgg

SEQ ID NO: 7 - DNA, 1214 bp, artificial sequence
  (spe) EF1alpha prom
actagtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaagcttctagagatccctcgac

SEQ ID NO: 8 - DNA, 1296 bp, artificial sequence
  (xho) Core 2 beta 1-6 GlcNAc transferase 1
ctcgagaccatgctgaggacgttgctgcgaaggagacttttttcttatcccaccaaatactactttatggttcttgttttatccctaatcaccttctccgttttaaggattcatcaaaagcctgaatttgtaagtgtcagacacttggagcttgctggggagaatcctagtagtgatattaattgcaccaaagttttacagggtgatgtaaatgaaatccaaaaggtaaagcttgagatcctaacagtgaaatttaaaaagcgccctcggtggacacctgacgactatataaacatgaccagtgactgttcttctttcatcaagagacgcaaatatattgtagaaccccttagtaaagaagaggcggagtttccaatagcatattctatagtggttcatcacaagattgaaatgcttgacaggctgctgagggccatctatatgcctcagaatttctattgcgttcatgtggacacaaaatccgaggattcctatttagctgcagtgatgggcatcgcttcctgttttagtaatgtctttgtggccagccgattggagagtgtggtttatgcatcgtggagccgggttcaggctgacctcaactgcatgaaggatctctatgcaatgagtgcaaactggaagtacttgataaatctttgtggtatggattttcccattaaaaccaacctagaaattgtcaggaagctcaagttgttaatgggagaaaacaacctggaaacggagaggatgccatcccataaagaagaaaggtggaagaagcggtatgaggtcgttaatggaaagctgacaaacacagggactgtcaaaatgcttcctccactcgaaacacctctcttttctggcagtgcctacttcgtggtcagtagggagtatgtggggtatgtactacagaatgaaaaaatccaaaagttgatggagtgggcacaagacacatacagccctgatgagtatctctgggccaccatccaaaggattcctgaagtcccgggctcactccctgccagccataagtatgatctatctgacatgcaagcagttgccaggtttgtcaagtggcagtactttgagggtgatgtttccaagggtgctccctacccgccctgcgatggagtccatgtgcgctcagtgtgcattttcggagctggtgacttgaactggatgctgcgcaaacaccacttgtttgccaataagtttgacgtggatgttgacctctttgccatccagtgtttggatgagcatttgagacacaaagctttggagacattaaaacactga

SEQ ID NO: 9 - DNA, 130 bp, artificial sequence
  (not) IgG1 hinge/CH2 intron
gcggccgccgcaggtaagccagcccaggcctcgccctccagctcaaggcgggacaggtgccctagagtagcctgcatccagggacaggccccagccgggtgctgacacgtccacctccatctcttcctca

SEQ ID NO: 10 - DNA, 133 bp, artificial sequence
  (hpa1) SV40 poly A
gttaacttgtttattgcagcttataatggttacaaataaagcaatagcatcacaaatttcacaaataaagcatttttttcactgcattctagttgtggtttgtccaaactcatcaatgtatcttatcatgtct

SEQ ID NO: 11 - DNA, 805 bp, artificial sequence
  (bamh1) neomycin (rev)
ggatcctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatggtc

SEQ ID NO: 12 - DNA, 232 bp, artificial sequence
  (pst) HSV1 tk promoter -215 to +19, with G to A mutation at +7
ctgcagagtcgctcggtgttcgaggccacacgcgtcaccttaatatgcgaagtggacctgggaccgcgccgccccgactgcatctgcgtgttcgaattcgccaatgacaagacgctgggcggggtttgtgtcatcatagaactaaagacatgcaaatatatttcttccggggacaccgccagcaaacgcgagcaacgggccacggggatgaagcagctgcgccactccctga

SEQ ID NO: 13 - DNA, 182 bp, artificial sequence
  (bg12) SV40 origin (minus enhancer)
agatctcccgcccctaactccgcccatcccgcccctaactccgcccagttccgcccattctccgccccatggctgactaattttttttatttatgcagaggccgaggccgcggcctctgagctattccagaagtagtgaggaggcttttttggaggcctaggcttttgcaaaaagctaattc

SEQ ID NO: 14 - DNA, 4930 bp, artificial sequence
  Porcine alpha 1,3 Galactosyltransferase Expression Vector
ggcgtaatctgctgcttgcaaacaaaaaaaccaccgctaccagcggtggtttgtttgccggatcaagagctaccaactctttttccgaaggaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgccgaattaccgcggtctttcggacttttgaaagtgatggtggtgggggaaggattcgaaccttcgaagtcgatgacggcagatttagagtctgctccctttggccgctcgggaaccccaccacgggtaatgcttttactggcctgctcccttatcgggaagcggggcgcatcatatcaaatgacgcgccgctgtaaagtgttacgttgagaaagctgctccctgcttgtgtgttggaggtcgctgagtagtgcgcgagtaaaatttaagctacaacaaggcaaggcttgaccgacaattgcatgaagaatctgcttagggttaggcgttttgcgctgcttcggactagtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaagcttaccatgaatgtcaaaggaagagtggttctgtcaatgctgcttgtctcaactgtaatggttgtgttttgggaatacatcaacagaaacccagaagttggcagcagtgctcagaggggctggtggtttccgagctggtttaacaatgggactcacagttaccacgaagaagaagacgctataggcaacgaaaaggaacaaagaaaagaagacaacagaggagagcttccgctagtggactggtttaatcctgagaaacgcccagaggtcgtgaccataaccagatggaaggctccagtggtatgggaaggcacttacaacagagccgtcttagataattattatgccaaacagaaaattaccgtgggcttgacggtttttgctgtcggaagatacattgagcattacttggaggagttcttaatatctgcaaatacatacttcatggttggccacaaagtcatcttttacatcatggtggatgatatctccaggatgcctttgatagagctgggtcctctgcgttcctttaaagtgtttgagatcaagtccgagaagaggtggcaagacatcagcatgatgcgcatgaagaccatcggggagcacatcctggcccacatccagcacgaggtggacttcctcttctgcattgacgtggatcaggtcttccaaaacaactttggggtggagaccctgggccagtcggtggctcagctacaggcctggtggtacaaggcacatcctgacgagttcacctacgagaggcggaaggagtccgcagcctacattccgtttggccagggggatttttattaccacgcagccatttttgggggaacacccactcaggttctaaacatcactcaggagtgcttcaagggaatcctccaggacaaggaaaatgacatagaagccgagtggcatgatgaaagccatctaaacaagtatttccttctcaacaaacccactaaaatcttatccccagaatactgctgggattatcatataggcatgtctgtggatattaggattgtcaagatagcttggcagaaaaaagagtataatttggttagaaataacatctgagcggccgccgcaggtaagccagcccaggcctcgccctccagctcaaggcgggacaggtgccctagagtagcctgcatccagggacaggccccagccgggtgctgacacgtccacctccatctcttcctcagttaacttgtttattgcagcttataatggttacaaataaagcaatagcatcacaaatttcacaaataaagcatttttttcactgcattctagttgtggtttgtccaaactcatcaatgtatcttatcatgtctggatccgctagcgctttattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatcgcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatggtggcctgcagagtcgctcggtgttcgaggccacacgcgtcaccttaatatgcgaagtggacctgggaccgcgccgccccgactgcatctgcgtgttcgaattcgccaatgacaagacgctgggcggggtttgtgtcatcatagaactaaagacatgcaaatatatttcttccggggacaccgccagcaaacgcgagcaacgggccacggggatgaagcagctgcgccactccctgaagatctcccgcccctaactccgcccatcccgcccctaactccgcccagttccgcccattctccgccccatggctgactaattttttttatttatgcagaggccgaggccgcggcctctgagctattccagaagtagtgaggaggcttttttggaggcctaggcttttgcaaaaagctaattc

SEQ ID NO: 15 - DNA, 593 bp, artificial sequence; identical to SEQ ID NO: 5
  pMB1 origin (pBR322 ori)

SEQ ID NO: 16 - DNA, 332 bp, artificial sequence; identical to SEQ ID NO: 6
  (sac2) synthetic tyrosine suppressor tRNA gene (supF gene) remnant of ASV LTR

SEQ ID NO: 17 - DNA, 1192 bp, artificial sequence
  (spe) EF1alpha prom
actagtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaa

SEQ ID NO: 18 - DNA, 1089 bp, artificial sequence
  (hind3) porcine alpha 1,3 galactosyltransferase
aagcttaccatgaatgtcaaaggaagagtggttctgtcaatgctgcttgtctcaactgtaatggttgtgttttgggaatacatcaacagaaacccagaagttggcagcagtgctcagaggggctggtggtttccgagctggtttaacaatgggactcacagttaccacgaagaagaagacgctataggcaacgaaaaggaacaaagaaaagaagacaacagaggagagcttccgctagtggactggtttaatcctgagaaacgcccagaggtcgtgaccataaccagatggaaggctccagtggtatgggaaggcacttacaacagagccgtcttagataattattatgccaaacagaaaattaccgtgggcttgacggtttttgctgtcggaagatacattgagcattacttggaggagttcttaatatctgcaaatacatacttcatggttggccacaaagtcatcttttacatcatggtggatgatatctccaggatgcctttgatagagctgggtcctctgcgttcctttaaagtgtttgagatcaagtccgagaagaggtggcaagacatcagcatgatgcgcatgaagaccatcggggagcacatcctggcccacatccagcacgaggtggacttcctcttctgcattgacgtggatcaggtcttccaaaacaactttggggtggagaccctgggccagtcggtggctcagctacaggcctggtggtacaaggcacatcctgacgagttcacctacgagaggcggaaggagtccgcagcctacattccgtttggccagggggatttttattaccacgcagccatttttgggggaacacccactcaggttctaaacatcactcaggagtgcttcaagggaatcctccaggacaaggaaaatgacatagaagccgagtggcatgatgaaagccatctaaacaagtatttccttctcaacaaacccactaaaatcttatccccagaatactgctgggattatcatataggcatgtctgtggatattaggattgtcaagatagcttggcagaaaaaagagtataatttggttagaaataacatctga

SEQ ID NO: 19 - DNA, 130 bp, artificial sequence; identical to SEQ ID NO: 9
  (not) IgG1 hinge/CH2 intron

SEQ ID NO: 20 - DNA, 133 bp, artificial sequence; identical to SEQ ID NO: 10
  (hpa1) SV40 poly A

SEQ ID NO: 21 - DNA, 1034 bp, artificial sequence
  (bamh1) hygromycin b (rev)
ggatccgctagcgctttattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatcgcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggct

SEQ ID NO: 22 - DNA, 232 bp, artificial sequence; identical to SEQ ID NO: 12
  (pst) HSV1 tk promoter -215 to +19, with G to A mutation at +7

SEQ ID NO: 23 - DNA, 182 bp, artificial sequence; identical to SEQ ID NO: 13
  (bgl2) SV40 origin (minus enhancer)

SEQ ID NO: 24 - DNA, 5204 bp, artificial sequence
  Human PSGL-1 Expression vector
ggcgtaatctgctgcttgcaaacaaaaaaaccaccgctaccagcggtggtttgtttgccggatcaagagctaccaactctttttccgaaggaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgccgaattaccgcggtctttcggacttttgaaagtgatggtggtgggggaaggattcgaaccttcgaagtcgatgacggcagatttagagtctgctccctttggccgctcgggaaccccaccacgggtaatgcttttactggcctgctcccttatcgggaagcggggcgcatcatatcaaatgacgcgccgctgtaaagtgttacgttgagaaagctgctccctgcttgtgtgttggaggtcgctgagtagtgcgcgagtaaaatttaagctacaacaaggcaaggcttgaccgacaattgcatgaagaatctgcttagggttaggcgttttgcgctgcttcggactagtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaaaagcttctagagatccctcgacctcgagatccattgtgctctaaaggagatacccggccagacaccctcacctgcggtgcccagctgcccaggctgaggcaagagaaggccagaaaccatgcccatggggtctctgcaaccgctggccaccttgtacctgctggggatgctggtcgcttccgtgctagcgcagctgtgggacacctgggcagatgaagccgagaaagccttgggtcccctgcttgcccgggaccggagacaggccaccgaatatgagtacctagattatgatttcctgccagaaacggagcctccagaaatgctgaggaacagcactgacaccactcctctgactgggcctggaacccctgagtctaccactgtggagcctgctgcaaggcgttctactggcctggatgcaggaggggcagtcacagagctgaccacggagctggccaacatggggaacctgtccacggattcagcagctatggagatacagaccactcaaccagcagccacggaggcacagaccactccactggcagccacagaggcacagacaactcgactgacggccacggaggcacagaccactccactggcagccacagaggcacagaccactccaccagcagccacggaagcacagaccactcaacccacaggcctggaggcacagaccactgcaccagcagccatggaggcacagaccactgcaccagcagccatggaagcacagaccactccaccagcagccatggaggcacagaccactcaaaccacagccatggaggcacagaccactgcaccagaagccacggaggcacagaccactcaacccacagccacggaggcacagaccactccactggcagccatggaggccctgtccacagaacccagtgccacagaggccctgtccatggaacctactaccaaaagaggtctgttcatacccttttctgtgtcctctgttactcacaagggcattcccatggcagccagcaatttgtccgtcaactacccagtgggggccccagaccacatctctgtgaagcaggatcccgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagagaattacaacagtactgtccgggtggtcagcaccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactataaggacaccgcaccagtcctggactctgacggttcttacttcatatatagcaagctcaatatgaaaacaagcaagtgggagaaaacagattccttctcatgcaacgtgagacacgagggtctgaaaaattactacctaaagaagaccatctcccggtctccgggtaaatgagcggccgccgcaggtaagccagcccaggcctcgccctccagctcaaggcgggacaggtgccctagagtagcctgcatccagggacaggccccagccgggtgctgacacgtccacctccatctcttcctcagttaacttgtttattgcagcttataatggttacaaataaagcaatagcatcacaaatttcacaaataaagcatttttttcactgcattctagttgtggtttgtccaaactcatcaatgtatcttatcatgtctggatccgctagcgcttcaggcaccgggcttgcgggtcatgcaccaggtcgcgcggtccttcgggcactcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtggcctgcagagtcgctcggtgttcgaggccacacgcgtcaccttaatatgcgaagtggacctgggaccgcgccgccccgactgcatctgcgtgttcgaattcgccaatgacaagacgctgggcggggtttgtgtcatcatagaactaaagacatgcaaatatatttcttccggggacaccgccagcaaacgcgagcaacgggccacggggatgaagcagctgcgccactccctgaagatctcccgcccctaactccgcccatcccgcccctaactccgcccagttccgcccattctccgccccatggctgactaattttttttatttatgcagaggccgaggccgcggcctctgagctattccagaagtagtgaggaggcttttttggaggcctaggcttttgcaaaaagctaattc

SEQ ID NO: 25 - DNA, 593 bp, artificial sequence; identical to SEQ ID NO: 5
  pMB1 origin (pBR322 ori)

SEQ ID NO: 26 - DNA, 332 bp, artificial sequence; identical to SEQ ID NO: 6
  (sac2) synthetic tyrosine suppressor tRNA gene(supF gene)remnant of ASV LTR

SEQ ID NO: 27 - DNA, 1192 bp, artificial sequence; identical to SEQ ID NO: 17
  (spe) EF1alpha prom

SEQ ID NO: 28 - DNA, 1789 bp, artificial sequence
  (hind3) human PSGL-1/mouse IgG2b
aagcttctagagatccctcgacctcgagatccattgtgctctaaaggagatacccggccagacaccctcacctgcggtgcccagctgcccaggctgaggcaagagaaggccagaaaccatgcccatggggtctctgcaaccgctggccaccttgtacctgctggggatgctggtcgcttccgtgctagcgcagctgtgggacacctgggcagatgaagccgagaaagccttgggtcccctgcttgcccgggaccggagacaggccaccgaatatgagtacctagattatgatttcctgccagaaacggagcctccagaaatgctgaggaacagcactgacaccactcctctgactgggcctggaacccctgagtctaccactgtggagcctgctgcaaggcgttctactggcctggatgcaggaggggcagtcacagagctgaccacggagctggccaacatggggaacctgtccacggattcagcagctatggagatacagaccactcaaccagcagccacggaggcacagaccactccactggcagccacagaggcacagacaactcgactgacggccacggaggcacagaccactccactggcagccacagaggcacagaccactccaccagcagccacggaagcacagaccactcaacccacaggcctggaggcacagaccactgcaccagcagccatggaggcacagaccactgcaccagcagccatggaagcacagaccactccaccagcagccatggaggcacagaccactcaaaccacagccatggaggcacagaccactgcaccagaagccacggaggcacagaccactcaacccacagccacggaggcacagaccactccactggcagccatggaggccctgtccacagaacccagtgccacagaggccctgtccatggaacctactaccaaaagaggtctgttcatacccttttctgtgtcctctgttactcacaagggcattcccatggcagccagcaatttgtccgtcaactacccagtgggggccccagaccacatctctgtgaagcaggatcccgagcccagcgggcccatttcaacaatcaacccctgtcctccatgcaaggagtgtcacaaatgcccagctcctaacctcgagggtggaccatccgtcttcatcttccctccaaatatcaaggatgtactcatgatctccctgacacccaaggtcacgtgtgtggtggtggatgtgagcgaggatgacccagacgtccagatcagctggtttgtgaacaacgtggaagtacacacagctcagacacaaacccatagagagaattacaacagtactgtccgggtggtcagcaccctccccatccagcaccaggactggatgagtggcaaggagttcaaatgcaaggtcaacaacaaagacctcccatcacccatcgagagaaccatctcaaaaattaaagggctagtcagagctccacaagtatacatcttgccgccaccagcagagcagttgtccaggaaagatgtcagtctcacttgcctggtcgtgggcttcaaccctggagacatcagtgtggagtggaccagcaatgggcatacagaggagaactataaggacaccgcaccagtcctggactctgacggttcttacttcatatatagcaagctcaatatgaaaacaagcaagtgggagaaaacagattccttctcatgcaacgtgagacacgagggtctgaaaaattactacctaaagaagaccatctcccggtctccgggtaaatga

SEQ ID NO: 29 - DNA, 130 bp, artificial sequence; identical to SEQ ID NO: 9
  (not) IgG1 hinge/CH2 intron

SEQ ID NO: 30 - DNA, 133 bp, artificial sequence; identical to SEQ ID NO: 10
  (hpa1) SV40 poly A

SEQ ID NO: 31 - DNA, 621 bp, artificial sequence
  (bamh1) puromycin acetyltransferase
ggatccgctagcgcttcaggcaccgggcttgcgggtcatgcaccaggtcgcgcggtccttcgggcactcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccgggtcgacggtgtggcgcgtggcggggtagtcggcgaacgcggcggcgagggtgcgtacggcccgggggacgtcgtcgcgggtggcgaggcgcaccgtgggcttgtactcggtcatggtggc

SEQ ID NO: 32 - DNA, 232 bp, artificial sequence; identical to SEQ ID NO: 12
  (pst) HSV1 tk promoter -215 to +19, with G to A mutation at +7

SEQ ID NO: 33 - DNA, 182 bp, artificial sequence; identical to SEQ ID NO: 13
  (bgl2) SV40 origin (minus enhancer)

SEQ ID NO: 34 - protein, 10 aa, artificial sequence
  PSGL-1 consensus sequence
AXTTXXAXXE